(19) United States
(12) Patent Application Publication
(10) Pub. No.: US 2023/0390608 A1
(43) Pub. Date: Dec. 7, 2023

(54) SYSTEMS AND METHODS INCLUDING EAR-WORN DEVICES FOR VESTIBULAR REHABILITATION EXERCISES

(71) Applicant: Starkey Laboratories, Inc., Eden Prairie, MN (US)

(72) Inventors: Justin R. Burwinkel, Eden Prairie, MN (US); Penny A. Tyson, Edina, MN (US); Roy Rozenman, Mishmar David (IL)

(21) Appl. No.: 17/791,451

(22) PCT Filed: Jan. 8, 2021
(86) PCT No.: PCT/US2021/012743

§ 371 Date: Jul. 7, 2022

Related U.S. Application Data
(60) Provisional application No. 62/959,614, filed on Jan. 10, 2020.

(51) Int. Cl. A63B 26/00; A61B 5/00; G16H 20/30; A61B 5/024; A61B 5/021

(52) U.S. Cl. CPC A63B 26/003; A61B 5/4023; A61B 5/6815; G16H 20/30; A61B 5/02405; A61B 5/021

(57) ABSTRACT

Embodiments herein relate to systems including ear-worn devices that can be used to aid vestibular rehabilitation exercises for ear-worn device wearers. In an embodiment a vestibular rehabilitation management system is included having an ear-worn device including a control circuit, a microphone in communication with the control circuit, and a motion sensor in communication with the control circuit, wherein the vestibular rehabilitation management system is configured to evaluate a wearer of the ear-worn device, and select a vestibular training exercise based on the evaluation. Other embodiments are also included herein.

This application is being filed as a PCT International Patent application on Jan. 8, 2021, in the name of Starkey Laboratories, Inc., a U.S. national corporation, applicant for the designation of all countries, and Justin R. Burwinkel, a U.S. Citizen, and Penny A. Tyson, a U.S. Citizen, and Roy Rozenman, a Citizen of Israel, inventor(s) for the designation of all countries, and claims priority to U.S. Provisional Patent Application No. 62/959,614, filed Jan. 10, 2020, the contents of which are herein incorporated by reference in its entirety.

### FIELD

Embodiments herein relate to systems including ear-worn devices that can be used to aid vestibular rehabilitation exercises for ear-worn device wearers.

### BACKGROUND

The human balance system estimates body position and motion. Sensory inputs to the balance system include the vestibular signals from the inner ear (motion, equilibrium, spatial orientation), position sensation, visual signals, proprioception (touch) signals, and intended movement. These inputs are integrated by the vestibular nuclear complex which generates motor commands to drive the eyes and body. Loss of coordinated balance function can lead to impaired vision, disorientation, falls, and behavioral changes. Various individuals can benefit from vestibular/balance rehabilitation exercises. Vestibular rehabilitation exercises (also known in the art as “balance training exercises”) can improve primary and secondary balance system function by increasing musculoskeletal mass, strength, stamina and flexibility, facilitating sensory input integration compensation and habituation, and improving the individual's reaction(s) to balance destabilization. An important part of vestibular retraining therapy (VRT) is to establish an exercise program that can be performed regularly at home. Compliance with the home exercise program is essential to help the individual achieve rehabilitation goals.

### SUMMARY

Embodiments herein relate to systems including ear-worn devices that can be used to aid vestibular rehabilitation exercises for ear-worn device wearers. In a first aspect, a vestibular rehabilitation management system is included having an ear-worn device including a control circuit, a microphone in communication with the control circuit, and a motion sensor in communication with the control circuit, wherein the vestibular rehabilitation management system is configured to evaluate a wearer of the ear-worn device, and select a vestibular training exercise based on the evaluation.

In a second aspect, in addition to one or more of the preceding or following aspects, or in the alternative to some aspects, the vestibular training exercise selection includes selection of a dominant leg.

In a third aspect, in addition to one or more of the preceding or following aspects, or in the alternative to some aspects, the vestibular training exercise selection includes selection of a vestibular training exercise difficulty.

In a fourth aspect, in addition to one or more of the preceding or following aspects, or in the alternative to some aspects, the vestibular training exercise difficulty is selected with respect to a least one of a strength intensity index value, a vestibular function intensity index value, postural stability intensity index value, a reaction speed intensity index value, a stamina intensity index value, a speed intensity index value, a visual intensity index value, an environmental stimulation intensity index value, a proprioception intensity index value, and a cognitive load intensity index value.

In a fifth aspect, in addition to one or more of the preceding or following aspects, or in the alternative to some aspects, the vestibular rehabilitation management system is configured to select a vestibular training exercise based on a mode setting stored or accessed by the vestibular rehabilitation management system.

In a sixth aspect, in addition to one or more of the preceding or following aspects, or in the alternative to some aspects, the mode setting is selected from the group consisting of a training mode, a maintenance mode, and a daily life mode.

In a seventh aspect, in addition to one or more of the preceding or following aspects, or in the alternative to some aspects, the vestibular rehabilitation management system is configured to evaluate the wearer of the ear-worn device by sensing their movement over a time period preceding selection of the vestibular training exercise.

In an eighth aspect, in addition to one or more of the preceding or following aspects, or in the alternative to some aspects, movement is analyzed to detect at least one of swaying, stumbles, balance recovery, falls, and near-falls.

In a ninth aspect, in addition to one or more of the preceding or following aspects, or in the alternative to some aspects, the time period preceding selection of the vestibular training exercise spans about 1 minute to 4 weeks.

In a tenth aspect, in addition to one or more of the preceding or following aspects, or in the alternative to some aspects, the time period preceding selection of the vestibular training exercise includes a time period wherein the wearer of the ear-worn device is performing an exercise.

In an eleventh aspect, in addition to one or more of the preceding or following aspects, or in the alternative to some aspects, no vestibular training exercise is selected if detected movement crosses a threshold value.

In a twelfth aspect, in addition to one or more of the preceding or following aspects, or in the alternative to some aspects, the vestibular rehabilitation management system is further configured to present the selected vestibular training exercise to the wearer of the ear-worn device as a suggestion.

In a thirteenth aspect, in addition to one or more of the preceding or following aspects, or in the alternative to some aspects, the vestibular rehabilitation management system selects a time to present the selected vestibular training exercise to the wearer of the ear-worn device as a suggestion.

In a fourteenth aspect, in addition to one or more of the preceding or following aspects, or in the alternative to some aspects, the time is based on detected movement patterns of the wearer of the ear-worn device.

In a fifteenth aspect, in addition to one or more of the preceding or following aspects, or in the alternative to some aspects, the vestibular rehabilitation management system is further configured to present the selected vestibular training exercise to a care provider for the wearer of the ear-worn device as a suggestion.

In a sixteenth aspect, in addition to one or more of the preceding or following aspects, or in the alternative to some aspects, the vestibular rehabilitation management system is configured to evaluate the wearer of the ear-worn device by sensing their ability to complete a preceding exercise.

In a seventeenth aspect, in addition to one or more of the preceding or following aspects, or in the alternative to some aspects, the vestibular rehabilitation management system is configured to evaluate the wearer of the ear-worn device by sensing an accuracy of completion of a preceding exercise.

In an eighteenth aspect, in addition to one or more of the preceding or following aspects, or in the alternative to some aspects, the vestibular rehabilitation management system is configured to evaluate the wearer of the ear-worn device by determining the wearer's performance improvement over a series of preceding exercises.

In a nineteenth aspect, in addition to one or more of the preceding or following aspects, or in the alternative to some aspects, the vestibular rehabilitation management system is configured to evaluate the wearer of the ear-worn device by evaluating a health record input.

In a twentieth aspect, in addition to one or more of the preceding or following aspects, or in the alternative to some aspects, the vestibular rehabilitation management system is configured to evaluate the wearer of the ear-worn device by evaluating consumption of a medication.

In a twenty-first aspect, in addition to one or more of the preceding or following aspects, or in the alternative to some aspects, the vestibular rehabilitation management system is configured to evaluate the wearer of the ear-worn device by matching at least one of their age, gender, sex at birth, size or medical record with records stored in a database.

In a twenty-second aspect, in addition to one or more of the preceding or following aspects, or in the alternative to some aspects, the vestibular rehabilitation management system is configured to evaluate the wearer of the ear-worn device by evaluating answers to one or more questions posed to the ear-worn device wearer.

In a twenty-third aspect, in addition to one or more of the preceding or following aspects, or in the alternative to some aspects, the vestibular rehabilitation management system is configured to evaluate the wearer of the ear-worn device by evaluating measuring a degree of head or body sway with planar specificity.

In a twenty-fourth aspect, in addition to one or more of the preceding or following aspects, or in the alternative to some aspects, the vestibular rehabilitation management system is configured to evaluate the wearer of the ear-worn device by evaluating maximum lean before imbalance occurs.

In a twenty-fifth aspect, in addition to one or more of the preceding or following aspects, or in the alternative to some aspects, the vestibular rehabilitation management system is configured to evaluate the wearer of the ear-worn device by measuring a reaction speed of the wearer of the ear-worn device.

In a twenty-sixth aspect, in addition to one or more of the preceding or following aspects, or in the alternative to some aspects, the vestibular rehabilitation management system is configured to evaluate the wearer of the ear-worn device by evaluating imbalance recovery.

In a twenty-seventh aspect, in addition to one or more of the preceding or following aspects, or in the alternative to some aspects, the vestibular rehabilitation management system is configured to evaluate the wearer of the ear-worn device by evaluating imbalance recovery speed.

In a twenty-eighth aspect, in addition to one or more of the preceding or following aspects, or in the alternative to some aspects, evaluating imbalance recovery includes measuring a degree of head or body sway after imbalance.

In a twenty-ninth aspect, in addition to one or more of the preceding or following aspects, or in the alternative to some aspects, evaluating imbalance recovery includes measuring foot impact magnitude after imbalance.

In a thirtieth aspect, in addition to one or more of the preceding or following aspects, or in the alternative to some aspects, evaluating imbalance recovery includes measuring foot speed after imbalance.

In a thirty-first aspect, in addition to one or more of the preceding or following aspects, or in the alternative to some aspects, the vestibular rehabilitation management system is configured to evaluate the wearer of the ear-worn device by initiating imbalance and then evaluating imbalance recovery.

In a thirty-second aspect, in addition to one or more of the preceding or following aspects, or in the alternative to some aspects, the vestibular rehabilitation management system is configured to evaluate the wearer of the ear-worn device by detecting imbalance and then evaluating imbalance recovery.

In a thirty-third aspect, in addition to one or more of the preceding or following aspects, or in the alternative to some aspects, further can include an ear-worn device monitoring system in communication with the ear-worn device, the ear-worn device monitoring system can include a control circuit.

In a thirty-fourth aspect, in addition to one or more of the preceding or following aspects, or in the alternative to some aspects, the vestibular rehabilitation management system is configured to receive information from an accessory device.

In a thirty-fifth aspect, in addition to one or more of the preceding or following aspects, or in the alternative to some aspects, the information received from the accessory device can include movement data regarding the wearer of the ear-worn device.

In a thirty-sixth aspect, in addition to one or more of the preceding or following aspects, or in the alternative to some aspects, the movement data from the accessory device reflects movement of at least one of the wearer's arm, leg, or hip.

In a thirty-seventh aspect, in addition to one or more of the preceding or following aspects, or in the alternative to some aspects, movement data below a threshold value is indicative of the accessory device not being held.

In a thirty-eighth aspect, in addition to one or more of the preceding or following aspects, or in the alternative to some aspects, the vestibular rehabilitation management system is configured to record patterns of the accessory device being held and not being held.

In a thirty-ninth aspect, in addition to one or more of the preceding or following aspects, or in the alternative to some aspects, the vestibular rehabilitation management system is configured to pace exercises based on patterns of the accessory device being held and not being held.

In a fortieth aspect, in addition to one or more of the preceding or following aspects, or in the alternative to some aspects, the information received from the accessory device can include data derived from a camera.

In a forty-first aspect, in addition to one or more of the preceding or following aspects, or in the alternative to some aspects, the information received from the accessory device can include data regarding whether the wearer of the ear-worn device is using their arm for support.

In a forty-second aspect, in addition to one or more of the preceding or following aspects, or in the alternative to some aspects, the accessory device can include a personal communications device.

In a forty-third aspect, in addition to one or more of the preceding or following aspects, or in the alternative to some aspects, the accessory device can include a smart phone.

In a forty-fourth aspect, in addition to one or more of the preceding or following aspects, or in the alternative to some aspects, the vestibular rehabilitation management system is configured to receive information regarding an environment of the wearer of the ear-worn device from an accessory device.

In a forty-fifth aspect, in addition to one or more of the preceding or following aspects, or in the alternative to some aspects, the information regarding the environment of the wearer can include information regarding obstacles in the environment of the wearer of the ear-worn device.

In a forty-sixth aspect, in addition to one or more of the preceding or following aspects, or in the alternative to some aspects, the ear-worn device further can include a blood pressure sensor.

In a forty-seventh aspect, in addition to one or more of the preceding or following aspects, or in the alternative to some aspects, the ear-worn device further can include a heart rate sensor.

In a forty-eighth aspect, in addition to one or more of the preceding or following aspects, or in the alternative to some aspects, the ear-worn device is further configured to transmit data based on motion sensor data to an accessory device.

In a forty-ninth aspect, in addition to one or more of the preceding or following aspects, or in the alternative to some aspects, the ear-worn device includes a hearing aid.

In a fiftieth aspect, in addition to one or more of the preceding or following aspects, or in the alternative to some aspects, further can include a second ear-worn device.

In a fifty-first aspect, a vestibular rehabilitation management system is included having an ear-worn device can include a control circuit, a microphone in communication with the control circuit, and a motion sensor in communication with the control circuit, wherein the ear-worn device is configured to monitor signals from the motion sensor to detect system declared performance of an exercise, monitor signals from the microphone to detect wearer declared performance of an exercise, and compare system declared performance of the exercise with wearer declared performance of the exercise.

In a fifty-second aspect, in addition to one or more of the preceding or following aspects, or in the alternative to some aspects, the vestibular rehabilitation management system is configured to initiate a report to a third party if the system declared performance of the exercise and the wearer declared performance of the exercise deviates by at least a threshold value.

In a fifty-third aspect, in addition to one or more of the preceding or following aspects, or in the alternative to some aspects, the vestibular rehabilitation management system is configured to initiate a calibration process if the system declared performance of the exercise and the wearer declared performance of the exercise deviates by at least a threshold value.

In a fifty-fourth aspect, in addition to one or more of the preceding or following aspects, or in the alternative to some aspects, the system declared performance of an exercise is based on motion sensor signals crossing a threshold value.

In a fifty-fifth aspect, a vestibular rehabilitation management system is included having an ear-worn device can include a control circuit, a microphone in communication with the control circuit, and a motion sensor in communication with the control circuit, wherein the vestibular rehabilitation management system is configured to monitor signals from the microphone to detect a voice of a wearer of the ear-worn device.

In a fifty-sixth aspect, in addition to one or more of the preceding or following aspects, or in the alternative to some aspects, the vestibular rehabilitation management system is further configured to evaluate the signals from the microphone to detect spoken commands.

In a fifty-seventh aspect, in addition to one or more of the preceding or following aspects, or in the alternative to some aspects, the spoken commands selected from the group consisting of start exercise, pause exercise, stop exercise, and proceed with next exercise.

In a fifty-eighth aspect, in addition to one or more of the preceding or following aspects, or in the alternative to some aspects, the vestibular rehabilitation management system is further configured to evaluate the signals from the microphone to detect spoken commands and evaluate signals from the motion sensor to detect gesture commands.

In a fifty-ninth aspect, in addition to one or more of the preceding or following aspects, or in the alternative to some aspects, the vestibular rehabilitation management system is further configured to evaluate the signals from the microphone to detect a condition of the wearer of the ear-worn device.

In a sixtieth aspect, in addition to one or more of the preceding or following aspects, or in the alternative to some aspects, the vestibular rehabilitation management system is configured to select a vestibular training exercise based on the condition of the wearer of the ear-worn device.

In a sixty-first aspect, in addition to one or more of the preceding or following aspects, or in the alternative to some aspects, the vestibular rehabilitation management system is further configured to evaluate the signals from the microphone to detect an exertion state of the wearer of the ear-worn device.

In a sixty-second aspect, in addition to one or more of the preceding or following aspects, or in the alternative to some aspects, the vestibular rehabilitation management system is further configured to evaluate the signals from the microphone to detect a quality of speech of the wearer of the ear-worn device.

In a sixty-third aspect, in addition to one or more of the preceding or following aspects, or in the alternative to some aspects, the vestibular rehabilitation management system is further configured to evaluate the signals from the microphone to detect a distress state of the wearer of the ear-worn device.

In a sixty-fourth aspect, a vestibular rehabilitation management system is included having an ear-worn device can include a control circuit, a microphone in communication with the control circuit, and a sensor package in communication with the control circuit, wherein the vestibular rehabilitation management system is configured to initiate or detect performance of an exercise by a wearer of the ear-worn device, monitor signals from the sensor package to detect a performance status of an exercise, and send information regarding the exercise to a remote location.

In a sixty-fifth aspect, in addition to one or more of the preceding or following aspects, or in the alternative to some aspects, the vestibular rehabilitation management system is further configured to initiate a real-time connection with a third party.

In a sixty-sixth aspect, in addition to one or more of the preceding or following aspects, or in the alternative to some aspects, the vestibular rehabilitation management system is further configured to receive input from a third party regarding the exercise or a future exercise.

In a sixty-seventh aspect, in addition to one or more of the preceding or following aspects, or in the alternative to some aspects, the vestibular rehabilitation management system sends information regarding the exercise to the remote location if the performance status represents a change over a previous performance status crossing a threshold value.

In a sixty-eighth aspect, a vestibular rehabilitation management system is included having a first ear-worn device can include a first control circuit, a first power control circuit in communication with the first control circuit, the first power control circuit can include a first battery, a first microphone in communication with the first control circuit, and a first motion sensor in communication with the first control circuit, and a second ear-worn device can include a second control circuit, a second power control circuit in communication with the second control circuit, the second power control circuit can include a second battery, a second microphone in communication with the first control circuit, and a second motion sensor in communication with the first control circuit, and wherein the vestibular rehabilitation management system is configured to initiate or detect initiation of an exercise, turn on the first motion sensor and monitor a signal from the first motion sensor, turn off the first motion sensor, turn on the second motion sensor, and monitor a signal from the second motion sensor.

In a sixty-ninth aspect, in addition to one or more of the preceding or following aspects, or in the alternative to some aspects, the vestibular rehabilitation management system is configured to turn off the first motion sensor, turn on the second motion sensor, and monitor a signal from the second motion sensor based on a time schedule.

In a seventieth aspect, in addition to one or more of the preceding or following aspects, or in the alternative to some aspects, the vestibular rehabilitation management system is configured to turn off the first motion sensor, turn on the second motion sensor, and monitor a signal from the second motion sensor based on a comparison of a remaining stored charge of the first battery versus a remaining stored charge of the second battery.

In a seventy-first aspect, in addition to one or more of the preceding or following aspects, or in the alternative to some aspects, the first motion sensor and the second motion sensor can include a gyroscope.

In a seventy-second aspect, a vestibular rehabilitation management system is included having a first ear-worn device can include a first control circuit, a first power control circuit in communication with the first control circuit, the first power control circuit can include a first battery, a first microphone in communication with the first control circuit, and a first motion sensor in communication with the first control circuit, and a second ear-worn device can include a second control circuit, a second power control circuit in communication with the second control circuit, the second power control circuit can include a second battery, a second microphone in communication with the first control circuit, and a second motion sensor in communication with the first control circuit, and wherein the vestibular rehabilitation management system is configured to initiate or detect initiation of an exercise, select either the first ear-worn device or the second ear-worn device, turn on the motion sensor of the selected device and/or execute wireless radio operations with the selected device, turn off the motion sensor of the non-selected device and/or cease or reduce wireless radio operations of the non-selected device.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a device wearer and components of a vestibular rehabilitation management system in accordance with various embodiments herein.

FIG. 2 is a schematic view of an accessory device in accordance with various embodiments herein.

FIG. 3 is a schematic view of a device wearer and components of a vestibular rehabilitation management system in accordance with various embodiments herein.

FIG. 4 is a schematic view of a device wearer and components of a vestibular rehabilitation management system in accordance with various embodiments herein.

FIG. 5 is a schematic view of a device wearer and components of a vestibular rehabilitation management system in accordance with various embodiments herein.

FIG. 6 is a schematic view of a device wearer and components of a vestibular rehabilitation management system in accordance with various embodiments herein.

FIG. 7 is a chart of exercise performance over time in accordance with various embodiments herein.

FIG. 8 is a chart of exercise performance over time in accordance with various embodiments herein.

FIG. 9 is a block diagram of dimensions of exercise difficulty in accordance with various embodiments herein.

FIG. 10 is a block diagram of different exercise modes in accordance with various embodiments herein.

FIG. 11 is a block diagram of aspects of an evaluation operation in accordance with various embodiments herein.

FIG. 12 is a schematic view of an accessory device in accordance with various embodiments herein.

FIG. 13 is a schematic view of a device wearer and components of a vestibular rehabilitation management system in accordance with various embodiments herein.

FIG. 14 is a schematic view of a device wearer and components of a vestibular rehabilitation management system in accordance with various embodiments herein.

FIG. 15 is a view of a vestibular rehabilitation management system in accordance with various embodiments herein.

FIG. 16 is a schematic diagram of a pair of ear-worn devices trading off in sensor operation in accordance with various embodiments herein.

FIG. 17 is a schematic view of an ear-worn device in accordance with various embodiments herein.

FIG. 18 is a schematic view of an ear-worn device as worn by a wearer in accordance with various embodiments herein.

FIG. 19 is a schematic view of components of an ear-worn device in accordance with various embodiments herein.

FIG. 20 is a schematic view of components of an accessory device in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, many individuals can benefit from vestibular rehabilitation exercises. In particular, many individuals who wear ear-worn devices (including, but not limited to, hearing assistance devices) can benefit from vestibular rehabilitation exercises. However, determining an appropriate starting level for an individual and then monitoring and advancing their progression can be challenging. Embodiments herein can be useful for determining an appropriate starting level. Embodiments herein can also be useful for monitoring the vestibular rehabilitation exercises undertaken by device wearers.

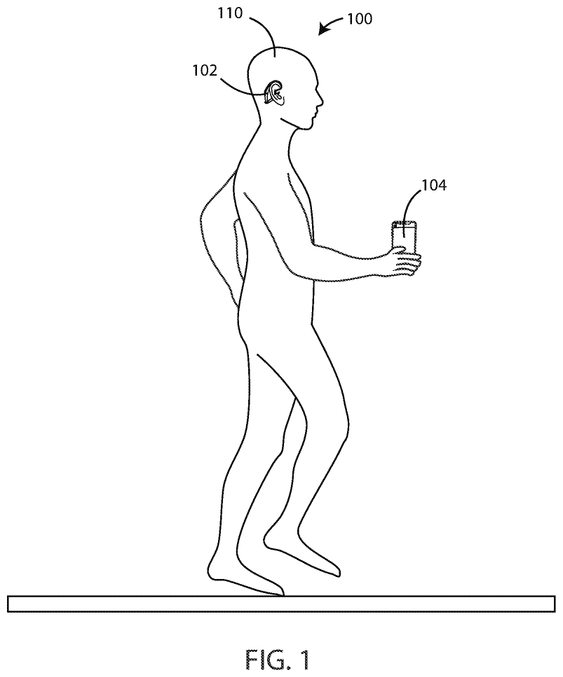

Referring now to FIG. 1, a schematic view of a device wearer 110 and components of a vestibular rehabilitation management system 100 are shown in accordance with various embodiments herein. The vestibular rehabilitation management system 100 includes an ear-worn device 102. The vestibular rehabilitation management system 100 can also include an accessory device 104. In various embodiments, the vestibular rehabilitation management system 100 can be configured to exchange information with (e.g., from and/or to) the accessory device 104. In various embodiments, the ear-worn device 102 can be further configured to transmit data based on one or more sensors (such as a motion sensor data) to the accessory device 104. The ear-worn device 102 can include various components (described in greater detail below) such as a control circuit, a microphone in communication with the control circuit, and a motion sensor and/or sensor package in communication with the control circuit.

The vestibular rehabilitation management system 100 can be configured to evaluate a wearer of the ear-worn device 102 and/or select a vestibular training exercise based on an evaluation. The vestibular rehabilitation management system 100 can evaluate the wearer of the ear-worn device in various ways (described in greater detail below). In various embodiments, the vestibular rehabilitation management system 100 can be configured to evaluate the wearer of the ear-worn device 102 by sensing their movement over a time period preceding selection of the vestibular training exercise. In various embodiments, the vestibular rehabilitation management system 100 can be configured to evaluate the wearer of the ear-worn device 102 by sensing their ability to complete a preceding exercise. In various embodiments, the vestibular rehabilitation management system 100 can be configured to evaluate the wearer of the ear-worn device 102 by sensing an accuracy of completion of a preceding exercise. In various embodiments, the vestibular rehabilitation management system 100 can be configured to evaluate the wearer of the ear-worn device 102 by determining the wearer's performance improvement over a series of preceding exercises.

In various embodiments, the vestibular rehabilitation management system 100 can be configured to evaluate the wearer of the ear-worn device 102 by evaluating a health record input. Health records can be obtained in various ways including through a secure API request, stored with one or more devices herein, and/or by way of query of a database. In various embodiments, the vestibular rehabilitation management system 100 can be configured to evaluate the wearer of the ear-worn device 102 by evaluating consumption of a medication. In various embodiments, the vestibular rehabilitation management system 100 can be configured to evaluate the wearer of the ear-worn device 102 by matching them against patients or groups of patients with similar criteria such as age. In various embodiments, the vestibular rehabilitation management system 100 can be configured to evaluate the wearer of the ear-worn device 102 by evaluating answers to one or more questions posed to the ear-worn device 102 wearer.

In various embodiments, the vestibular rehabilitation management system 100 can be configured to select a vestibular training exercise. In various embodiments, the vestibular rehabilitation management system 100 can be configured to select a vestibular training exercise based on a mode setting (described further below) stored or accessed by the vestibular rehabilitation management system 100. In various embodiments, the vestibular rehabilitation management system 100 can be further configured to present the selected vestibular training exercise to the wearer of the ear-worn device 102 as a suggestion. In various embodiments, the vestibular rehabilitation management system 100 selects a time to present the selected vestibular training exercise to the wearer of the ear-worn device 102 as a suggestion. In various embodiments, the vestibular rehabilitation management system 100 can be further configured to present the selected vestibular training exercise to a care provider for the wearer of the ear-worn device 102 as a suggestion. In some embodiments the vestibular rehabilitation management system 100 selects a time to present the selected vestibular training exercise based on determination of a current activity classifier. In some embodiments the vestibular rehabilitation management system 100 selects a time to present the selected vestibular training exercise based on a historical pattern/machine learning derived pattern of previous activity classifications

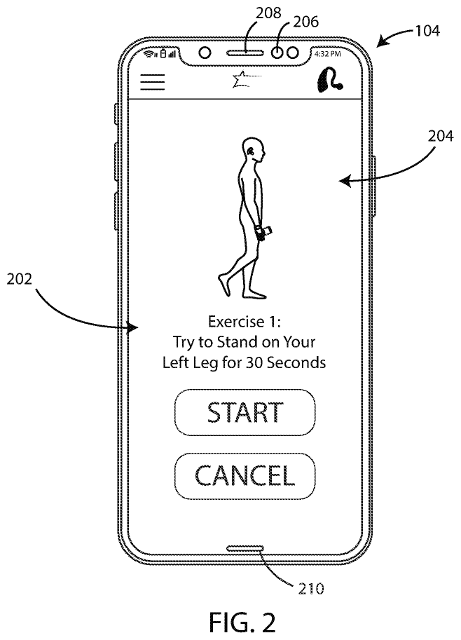

In various embodiments a vestibular rehabilitation management system can include and/or interface with an accessory device. Referring now to FIG. 2, a schematic view of an accessory device 104 is shown in accordance with various embodiments herein. The accessory device 104 can include a display screen 204. In some embodiments, the accessory device 104 can include a hologram display. The accessory device 104 can display various things visually 204, such as a suggestion 202 for exercise and/or instructions regarding the exercise. The accessory device 104 can also display feedback for the device wearer visually 204 such as how well they are performing the exercises, how far they have progressed through the exercises, and/or trends regarding the same.

The accessory device 104 can also include a camera 206. The camera 206 can be used to gather information regarding the device wearer, the environment around the device wearer, or both. The accessory device 104 can also include a speaker 208. The speaker 208 can be used to provide an audio interface with device wearer. The accessory device 104 can also include a microphone 210. The microphone 210 can be used to capture sound with respect to the device wearer and/or the environment of the device wearer. The accessory device 104 can also include one or more sensors such as one or more of those discussed below with regard to sensors of ear-worn devices.

In various embodiments, the vestibular rehabilitation management system 100 can be configured to present a selected vestibular training exercise to the wearer of the ear-worn device 102 as a suggestion 202. In some cases, the vestibular rehabilitation management system 100 can present a visual image of the selected exercise on the display screen 204.

In various embodiments, the vestibular rehabilitation management system 100 can select a time to present the selected vestibular training exercise to the wearer of the ear-worn device 102 as a suggestion 202. In various embodiments, the time can be based on preferences of the wearer of the ear-worn device 102 as input into the system. For example, the wearer can input into or otherwise indicate to the system that they prefer to exercise between the hours of 7 and 9 AM. In various embodiments, the time can be based on detected movement patterns of the wearer of the ear-worn device 102. For example, when the vestibular rehabilitation management system 100 detects a period of relatively little movement (such as by detecting that motions sensors indicate movement (or an averaged movement value) crossing below a threshold value then the system can suggest an exercise to the wearer of the ear-worn device. As another example, when sensors associated with one or more devices of the system detect a (pre-specified) period of 10, 15, 20, 30, or 60 min of sedentary time (or an amount of time falling within a range between any of the foregoing) then the system can suggest an exercise to the wearer of the ear-worn device. As another example, when the vestibular rehabilitation management system 100 detects a user activity substantially similar to activities of daily living that have been determined to be suitable for adaptation into exercises that challenge balance and load their muscles, such as standing on one leg while tooth brushing, taking two steps up while stair climbing, performing partial-squats while dishwasher loading, hopping or jumping while walking, and the like, then the system can suggest an exercise to the wearer of the ear-worn device and/or such detected user activities can be counted as exercises performed in accordance with various embodiments herein.

Suggestions can be responded to by the wearer of the ear-worn device. In some embodiments, the wearer can interface with the system in order to indicate that they will attempt the suggested exercise. In some embodiments, the wearer can interface with the system to indicate that they want to skip the suggested exercise. In some embodiments, the system can automatically detect that the user is attempting the suggested exercise.

In some embodiments, the system can provide audio feedback to the wearer of the ear-worn device (via the ear-worn device, the accessory device, and/or another device) to indicate/confirm that the exercise is ongoing and/or indicate/confirm that the exercise has begun. The audio feedback can take many different forms including, but not limited to, music, a beat, a metronome sound, words, or other sounds.

In various embodiments, the vestibular rehabilitation management system 100 can be configured to receive information from an accessory device 104. In this way, the accessory device 104 can be used in order to gather more information than might otherwise be available if only the ear-worn device was gathering information regarding the wearer of the ear-worn device. In various embodiments, the information received from the accessory device 104 can include movement data regarding the wearer of the ear-worn device 102. In various embodiments, the movement data from the accessory device 104 can reflect movement of at least one of the wearer's arm, leg, or hip. For example, if the accessory device is being held in the hand of the ear-worn device wearer, or worn on the wrist of the ear-worn device wearer, then movement data generated from movement sensors thereon can reflect movement of the arm which may or may not be consistent with movement data as gathered by the ear-worn devices which can be directly reflective of movement of the head. Similarly, if the accessory device is being worn on or about the waist of the ear-worn device wearer, then movement data generated from movement sensors thereon can reflect movement of the hip. In various embodiments, the information received from the accessory device 104 can also include data derived from the camera 206. In various embodiments, the information received from the accessory device 104 can include data regarding whether the wearer of the ear-worn device 102 can be using their arm for support. For example, if the accessory device 104 is being worn on the wrist of the wearer of the ear-worn device or being held in their hand and movement data from the accessory device indicates that movement has fallen below a threshold value, then it can be inferred that the arm of the wearer associated with the accessory device is being used for support (such as by being placed upon or against a steady object). In some embodiments, one or more cameras of the accessory device may be used to determine if the accessory device has been placed, e.g., on a table top while the user frees their hands to help support themselves during performance of an exercise.

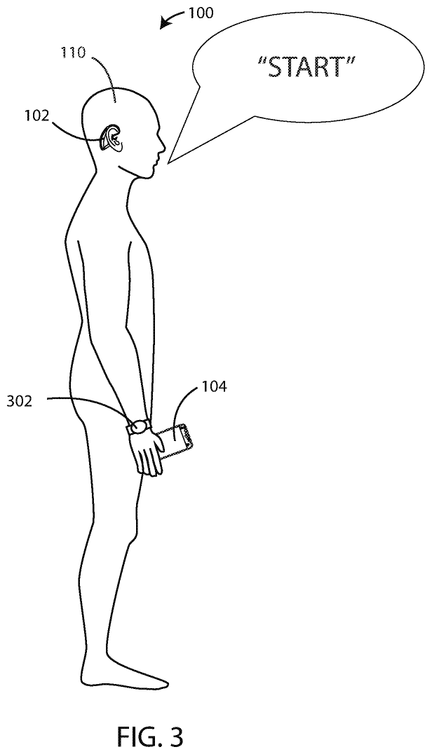

In various embodiments, the wearer of the ear-worn device can interface with the system through sound, touch, gestures, visually or the like. Referring now to FIG. 3, a schematic view of a device wearer 110 and components of a vestibular rehabilitation management system 100 is shown in accordance with various embodiments herein. As before, the vestibular rehabilitation management system 100 can include an ear-worn device 102. The vestibular rehabilitation management system 100 can also include an accessory device 104. In some cases, the vestibular rehabilitation management system 100 can also include and/or receive data from an additional device 302. The type of the additional device 302 is not particularly limited but can be various things such as a smart watch, a wearable monitoring device, or the like.

In various embodiments, the vestibular rehabilitation management system 100 can be configured to monitor signals from at least one microphone, own-voice sensor, or other vibration sensor to detect a voice of a wearer of the ear-worn device 102. As used herein, the term “microphone” shall include reference to all types of devices used to capture sounds including various types of microphones (including, but not limited to, carbon microphones, fiber optic microphones, dynamic microphones, electret microphones, ribbon microphones, laser microphones, condenser microphones, cardioid microphones, crystal microphones) and vibration sensors (including, but not limited to accelerometers and various types of pressure sensors). Microphones herein can include analog and digital microphones. Systems herein can also include various signal processing chips and components such as analog-to-digital converters and digital-to-analog converters. Systems herein can operate with audio data that is gathered, transmitted, and/or processed reflecting various sampling rates. By way of example, sampling rates used herein can include 8,000 Hz, 11,025 Hz, 16,000 Hz, 22,050 Hz, 32,000 Hz, 37,800 Hz, 44,056 Hz, 44,100 Hz, 47,250 Hz, 48,000 Hz, 50,000 Hz, 50,400 Hz, 64,000 Hz, 88,200 Hz, 96,000 Hz, 176,400 Hz, 192,000 Hz, or higher or lower, or within a range falling between any of the foregoing. Audio data herein can reflect various bit depths including, but not limited to 8, 16, and 24-bit depth.

The microphone or other vibration sensor can one associated with the ear-worn device 102, an accessory device 104, an additional device 302, or the like. In various embodiments, the vestibular rehabilitation management system 100 can be further configured to evaluate the signals from the microphone to detect spoken commands. In various embodiments, the spoken commands are not particularly limited but can include one or more commands such as “start exercise”, “pause exercise”, “stop exercise”, and “proceed with next exercise”, or equivalents thereof. In various embodiments, the vestibular rehabilitation management system 100 can be further configured to evaluate the signals from the microphone to both detect spoken commands and also evaluate signals from a motion sensor and/or a camera to detect gesture commands. The gesture commands are not particularly limited but can include one or more commands such as “start exercise”, “pause exercise”, “stop exercise”, and “proceed with next exercise”, or equivalents thereof.

Spoken commands can be recognized by the system in various ways. In some embodiments, data from a microphone can be processed using a speech recognition API that can provide output including the words recognized within the microphone data. Sound representing speech can also be distinguished from general noise using various techniques as aided by signal processing algorithms and/or machine learning classification techniques, and can include aspects of phoneme recognition, frequency analysis, and evaluation of acoustic features such as those referenced below. In some embodiments, techniques for separating speech from background noise can be used including employing remote proximity microphones operatively connected to the system, spectral subtraction, Wiener filtering, and mean-square error estimation. Spectral subtraction subtracts the power spectral density of the estimated interference from that of the mixture. The Wiener filter estimates clean speech from the ratios of speech spectrum and mixture spectrum. Mean-square error estimation models speech and noise spectra as statistically independent Gaussian random variables and estimates clean speech accordingly.

In various embodiments, the vestibular rehabilitation management system 100 can be further configured to evaluate the signals from the microphone to detect a condition of the wearer of the ear-worn device 102. In various embodiments, the vestibular rehabilitation management system 100 can be configured to select a vestibular training exercise based, at least in part, on the condition of the wearer of the ear-worn device 102 as determined by evaluating signals from the microphone.

In various embodiments, the vestibular rehabilitation management system 100 can be further configured to evaluate the signals from the microphone to detect an exertion state of the wearer of the ear-worn device 102. In various embodiments, the vestibular rehabilitation management system 100 can be further configured to evaluate the signals from the microphone to detect an emotional state of the wearer of the ear-worn device 102. In various embodiments, the vestibular rehabilitation management system 100 can be further configured to evaluate the signals from the microphone to detect a quality of speech of the wearer of the ear-worn device 102. In various embodiments, the vestibular rehabilitation management system 100 can be further configured to evaluate the signals from the microphone to detect a distress state of the wearer of the ear-worn device 102. Techniques of evaluating the wellness of ear-worn device wearers are described in U.S. Pat. Appl. No. 62/800,227 entitled “EFFICIENT WELLNESS MEASUREMENT IN EAR-WEARABLE DEVICES”, the content of which is herein incorporated by reference.

In various embodiments, one or more of the ear-worn device 102, the accessory device 104 (and/or another device) can be configured to extract features from the signals from the microphone(s) representing speech of the ear-worn device wearer and transmit the extracted features to a separate system or device for analysis of ear-worn device wearer emotional and/or physical status and/or conduct the analysis themselves.

Features extracted herein (regardless of which device is performing the operation) can include low-level and high-level features. Features extracted herein can include, but are not limited to, low-level acoustic features including prosodic (such as fundamental frequency, speech rate, intensity, duration, energy, pitch, etc.), voice quality (such as formant frequency and bandwidth, jitter and shimmer, glottal parameter, etc.), spectral (such as spectrum cut-off frequency, spectrum centroid, correlation density and mel-frequency energy, etc.), cepstral (such a Mel-Frequency Cepstral Coefficients (MFCCs), Linear Prediction Cepstral Coefficients (LPCC), etc.), and the like. In some embodiments, the open source toolkit OpenSmile can be used in order to extract features from acoustic data.

Emotions and status evaluated herein can include classifying the detected state or emotion in various ways. In some embodiments, a device wearer's state or emotion can be classified as being energetic, neutral, tired or various degrees thereof. In some embodiments, a device wearer's state or emotion can be classified as being positive, neutral, negative or various degrees thereof. In some embodiments, a device wearer's state or emotion can be classified as neutral, happy, sad, angry, worried, scared, frustrated, or various degrees thereof. In some embodiments, in addition to or in replacement of other categorizations, a device wearer's state or emotion can be classified based on a level of detected stress. In some embodiments, a device wearer's state or emotion can be classified as highly stressed, stressed, normal stress, low stress, or various degrees thereof. In some embodiments, a discrete emotion description model can be used and in other embodiments a continuous emotion description model can be used. In some embodiments, a two-dimensional arousal-valence model can be used for classification. Many different specific classifications are contemplated herein.

Status evaluated herein is not limited to that derived through the evaluation of sound. As described below, devices herein can include sensors such as heart rate sensors, blood pressure sensors, cameras, and the like that can be used independently or in combination with microphone(s) in order to evaluate the emotion and/or physical status (including exertion) of the device wearer.

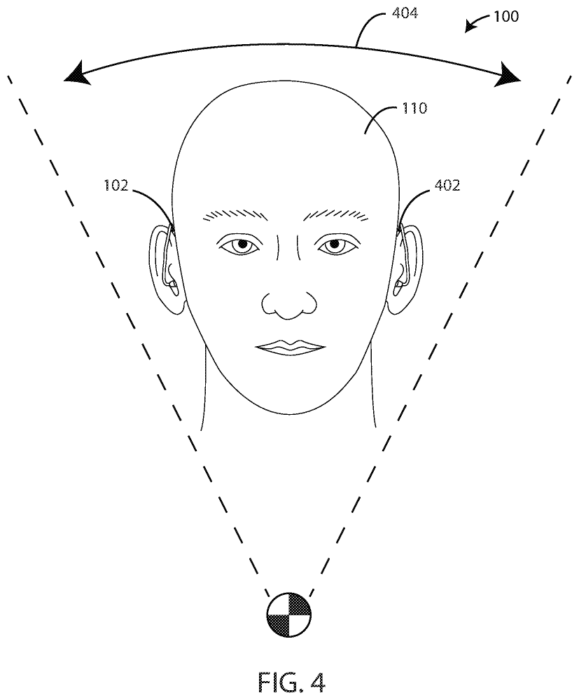

Movement can be used by systems herein for various purposes including, but not limited to, evaluating the ear-worn device wearer, monitoring exercises, and the like. Referring now to FIG. 4, a schematic view of a device wearer 110 and components of a vestibular rehabilitation management system 100 is shown in accordance with various embodiments herein. As before, the vestibular rehabilitation management system 100 includes an ear-worn device 102. In this example, the vestibular rehabilitation management system 100 also includes a second ear-worn device 402. The ear-worn device 102 and/or the second ear-worn device 402 can detect movement of the wearer of the ear-worn device 102 including lateral movement 404, such as by using a motion sensor and/or another type of sensor. In some cases, the lateral movement can be indicative of sway which may reflect the condition of the ear-worn device wearer. For example, a greater degree of observed sway may indicate a device wearer with lower level of balance system function. In some cases, the lateral movement can be indicative of a leaning action which may be done as part of an exercise or as part of an evaluation of the wearer of the ear-worn device. In some cases, the farther a device wearer can lean without losing their balance (as indicated by e.g., taking a recovery step, exhibiting excessive sway, falling or nearly falling, and the like) the better their balance system function is.

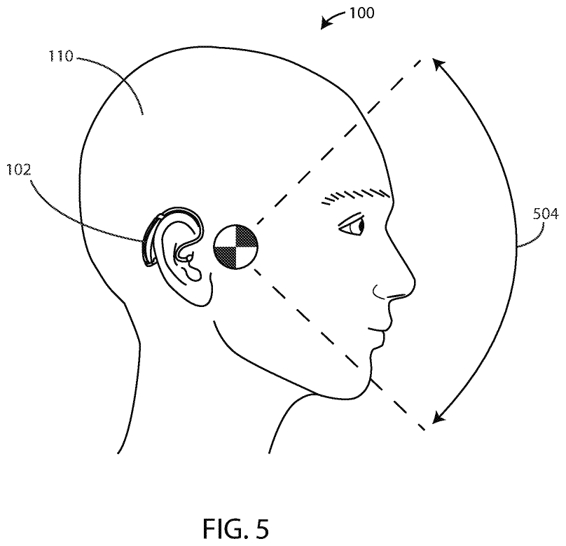

It will be appreciated, however, that in many cases herein movement detection is not limited to movement and/or rotation in only particular directions or planes. Referring now to FIG. 5, a schematic view of a device wearer 110 and components of a vestibular rehabilitation management system 100 is shown in accordance with various embodiments herein. As before, the vestibular rehabilitation management system 100 includes an ear-worn device 102. FIG. 5 shows a wearer 110 of the ear-worn device 102. The vestibular rehabilitation management system 100 can detect movement of the wearer 110 of the ear-worn device 102 includes vertical rotational movement 504 (e.g., rotation movement within a vertical plane).

In the context of performing an exercise, there can be a tendency for movement (including head movement) up and down as the device wearer is looking at an accessory device, such as a smart phone. Therefore, for purposes of monitoring performance of exercises, it can be useful to exclude, filter out, or otherwise deprioritize movement within a particular plane that is consistent with the device wearer's head tipping down in order to look at their accessory device or to otherwise view exercise instructions. As such, in various embodiments, the vestibular rehabilitation management system 100 can be configured to evaluate the wearer of the ear-worn device 102 by evaluating measuring a degree of head or body sway with planar specificity. Movement (such as head movement) consistent with the device wearer looking down at their accessory device and then back up again can be excluded, filtered out, or otherwise deprioritized.

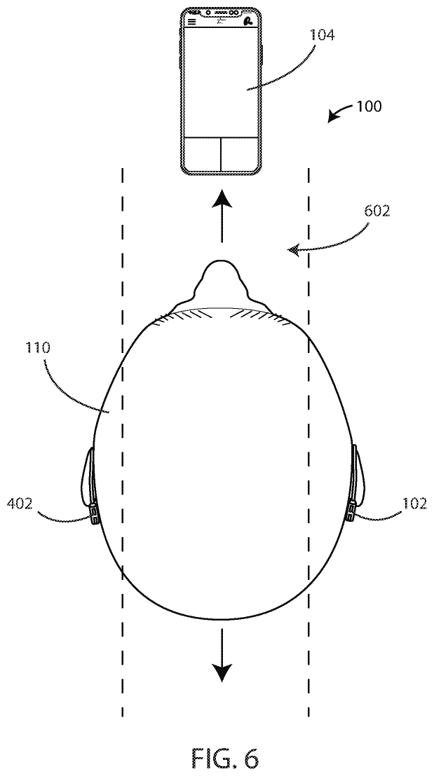

Referring now to FIG. 6, a schematic view of a device wearer 110 and components of a vestibular rehabilitation management system 100 is shown in accordance with various embodiments herein. As before, the vestibular rehabilitation management system 100 includes an ear-worn device 102. In this example, the vestibular rehabilitation management system 100 also includes or communicates with an accessory device 104. In this example, the vestibular rehabilitation management system 100 also includes a second ear-worn device 402. FIG. 6 illustrates a forward/backward plane 602. In various embodiments herein, movement of the head within the forward/backward plane 602 (such as movement consistent with tipping the head in the forward/backward plane 602) can be excluded, filtered out, or otherwise deprioritized in order to allow the system to more accurately detect movement which reflects execution of the exercise instead of movement which may only reflect the device wearer looking down at their accessory device and then back up again.

In many cases, it would be expected that the device wearer's performance of exercises (in various dimensions) should improve over time. In such a case, it can be important to increase the difficulty level of exercises over time to match the device wearer's performance such that the device wearer is continually challenged and does not become bored or disinterested. In many cases exercises get progressively more difficult in terms of: duration of exercise, repetitions, repetition sets, degree of movement (deep squats, more extreme limits of stability when leaning to side, front or back), etc. However, if improvement is not detected over time, then it can be important to not ramp up the difficulty level too quickly or too significantly so that the device wearer can keep up with the exercises. Further, while improvement over time is expected, detecting decreasing performance may be very important because it may indicate a decompensation of the device wearer and it may be valuable to alert a care provider to such decreasing performance.

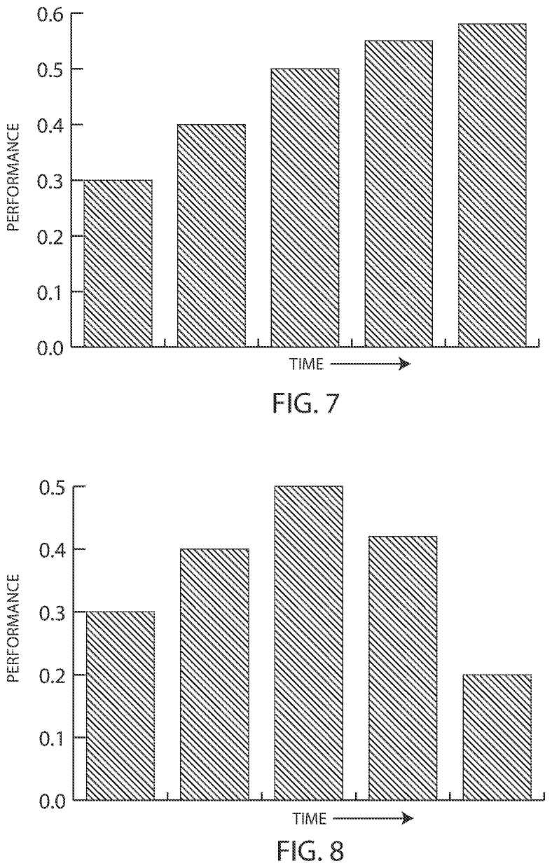

Referring now to FIG. 7, a chart of exercise performance over time is shown in accordance with various embodiments herein. This example shows a pattern of increasing performance over time. In various embodiments, the vestibular rehabilitation management system can be configured to evaluate the wearer of the ear-worn device by determining an exercise performance trend.

In various embodiments, the vestibular rehabilitation management system can be configured to evaluate the wearer of the ear-worn device by sensing their ability to complete a preceding exercise. In various embodiments, the vestibular rehabilitation management system 100 can be configured to evaluate the wearer of the ear-worn device by sensing an accuracy of completion of a preceding exercise. In some cases, the system can distinguish between attempts to perform exercises and successful, accurate completion of exercises. In some cases the system can provide feedback to the device wearer either at the end of each exercise or the end of each set and acknowledge one or both of successful completions and attempts. In some embodiments, the system can prompt the device wearer until a specific number of successful completions has been reached. In some embodiments, the system can prompt the device wearer until a specific number of combined successful completions and attempts has been reached.

Referring now to FIG. 8, a chart of exercise performance over time is shown in accordance with various embodiments herein. This example shows a pattern of decreasing performance over time. In various embodiments, the system can be configured to alert a third party, such as a care provider, if a pattern of decreasing performance over time is detected.

Performance can be assessed in various ways. In some embodiments, one or more aspects such as magnitude of rotation with respect to a threshold value, magnitude of movement with respect to a threshold value, magnitude of lean (such as without losing balance) with respect to a threshold value, duration of balance with respect to a threshold value, and the like can be considered when assessing the performance of the device wearer in performing exercises.

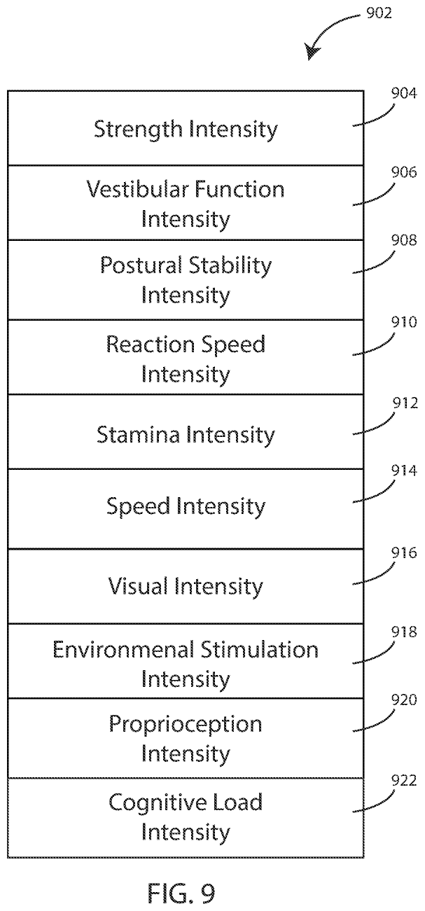

It will be appreciated that exercise difficulty can have various different dimensions. Referring now to FIG. 9, a block diagram of dimensions of exercise difficulty 902 is shown in accordance with various embodiments herein. The exercise difficulty 902 can include a strength intensity 904. The exercise difficulty 902 also can include a vestibular function intensity 906. The exercise difficulty 902 also can include a postural stability intensity 908 (which can include, for example, the strength/stability/movement of the ankle, hip, knee, etc.). The exercise difficulty 902 also can include a reaction speed intensity 910. The exercise difficulty 902 also can include a stamina intensity 912. The exercise difficulty 902 also can include a speed intensity 914. The exercise difficulty 902 also can include a visual intensity 916 (for example, conducting exercises with eyes open or closed and/or providing disrupting visual cues via a suitable device/technique such as smart glasses, holograms, artificial or augmented reality, or other types of displays). The exercise difficulty 902 also can include an environmental stimulation intensity (variation in, gradation of, or manipulation of environmental sound, light, etc.) 918. In some cases, such as for visual intensity and/or for environmental stimulation intensity a camera can be used to sense the level of ambient light and assign an intensity value or intensity index value based on detected ambient light. In some cases lighting within a room or environment can be controlled by the system to adjust ambient light levels (directly or indirectly and/or through IoT control techniques). Environmental sound intensity can be adjusted in various ways such as through control of devices making noise as well as through techniques such as adjusting a vent mechanism of an ear-worn device, wherein opening the vent allowed more environmental sound to reach the device wearer's ear drum. Thus, in various embodiments, the ear-worn device can activate an auto-vent feature that will actively close off a vent of the ear-worn device to provide greater acoustic separation between sounds in the ear canal of the device wearer (i.e., reaching the ear drum of the user and being heard by the device wearer) from the ambient sounds external to the ear coupling. Examples of auto-vent mechanisms/features include, but are not limited to, those found in commonly-owned U.S. Pat. No. 8,923,543, entitled HEARING ASSISTANCE DEVICE VENT VALVE, and commonly-owned U.S. Provisional Patent Application No. 62/850,805, entitled SOLENOID ACTUATOR IN A HEARING DEVICE, both of which are hereby incorporated by reference herein in their entirety. The exercise difficulty 902 also can include a proprioception intensity 920 (such as standing on a compliant surface—foam block, pillow, sand, balance board, balance ball, etc.—versus standing on solid ground). The exercise difficulty 902 also can include a cognitive load intensity 922 (such as where the instructions provided to the user are purposefully made more complex—increasing number of instructions provided at one time, more complex words, etc.—or asking the user to navigate around obstacles in their environment, or presenting the user with a dual task, such as having them respond to questions or perform other secondary tasks while performing an exercise).

In various embodiments, the exercise difficulty 902 can be selected with respect to a least one of a strength intensity 904 index value, a vestibular function intensity 906 index value, postural stability intensity 908 index value, a reaction speed intensity 910 index value, a stamina intensity 912 index value, a speed intensity 914 index value, a visual intensity 916 index value, an environmental stimulation intensity 918 index value, a proprioception intensity 920 index value, and a cognitive load intensity 922 index value. In various embodiments, the exercise difficulty 902 can be selected with respect to a least two of the preceding, three of the preceding, four of the preceding, five of the preceding, six of the preceding, seven of the preceding, eight of the preceding, nine of the preceding, or all ten of the preceding.

Different exercises can impact (or implicate) these different dimensions differently. For example, some exercises may depend more on strength in order to be able to complete them while other exercises may depend more on reaction speed. By calculating the performance of the device wearer across multiple exercises that are different with regard to how they implicate the difference dimensions, the system can determine the relative status of the device wearer with respect to strength, reaction speed, vestibular function, and the like.

In some embodiments, the system can intersperse exercises that are several levels too difficult for the device wearer (e.g., beyond their previous or current level) to give the device wearer a sense of being challenged, which may be helpful to excite the user or to otherwise maintain user engagement and compliance. This can be limited to certain types of exercises or difficulty types so as not the put the device wearer at risk for e.g., falling while performing the exercises.

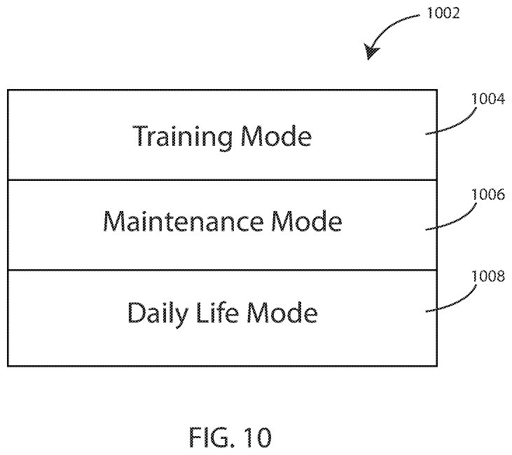

In some cases herein, exercise and exercise selections can depend on an exercise mode setting 1002. Referring now to FIG. 10, a block diagram of different exercise modes is shown in accordance with various embodiments herein. The mode setting 1002 includes can include a training mode 1004. In a training mode 1004 the series of exercises presented to the device wearer can ramp up in difficulty relatively quickly so as to maximize improvements obtained through vestibular rehabilitation exercises. The mode setting 1002 can also include a maintenance mode 1006. In a maintenance mode 1006 the series of exercises presented to the device wearer can ramp up more gradually or not at all in view of a device wearer that may simply be looking to maintain their current degree of balance system function. The mode setting 1002 can also include a daily life mode 1008. In a daily life mode 1008 the series of exercises can be tailored to and fit into the lifestyle of the device wearer. For example, if the system detects substantial movement throughout the day or otherwise detects that the device wearer is exhausted then the system may not suggest any exercises to the device wearer or may suggest only those with relatively low difficulty. In a further example, if system detects a user activity substantially similar activities of daily living which have been determined to be suitable for adaptation into an exercise, then the system may suggest to the user to perform said adapted activity of daily living as an exercise.

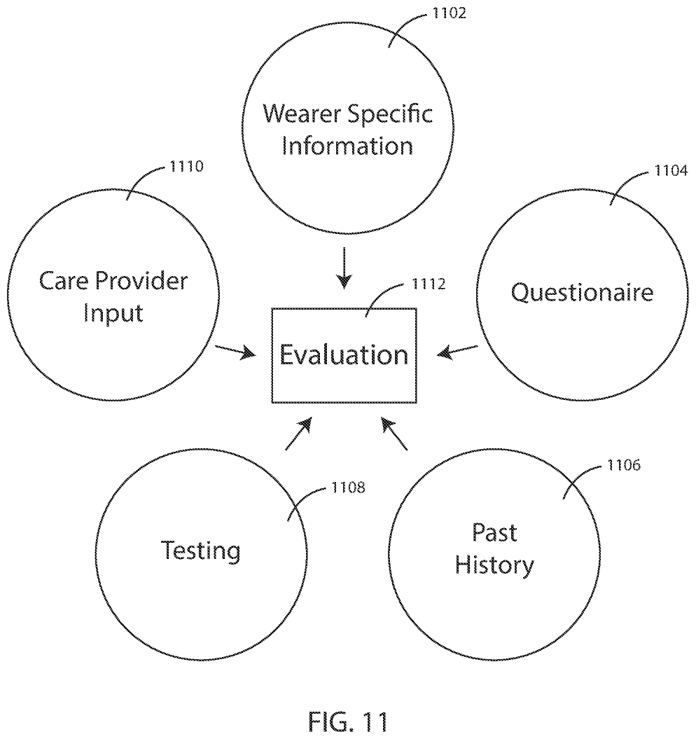

Aspects of evaluating a device wearer are described in greater detail below. However, referring now to FIG. 11, a block diagram of some aspects of an evaluation 1112 operation is shown in accordance with various embodiments herein. The ear-worn device wearer evaluation 1112 can include evaluating wearer specific information 1102. The ear-worn device wearer evaluation 1112 can also include presenting questions and/or a questionnaire 1104 to the ear-worn device wearer and/or a third party and then receiving answers to the same. As such, in various embodiments, the vestibular rehabilitation management system 100 can be configured to evaluate the wearer of the ear-worn device 102 by evaluating answers to one or more questions posed to the ear-worn device wearer 110.

The ear-worn device wearer evaluation 1112 can also include evaluating a past history 1106 of the ear-worn device wearer, such as evaluating the medical and/or drug records of the ear-worn device wearer. The ear-worn device wearer evaluation 1112 can also include performing testing 1108 on the ear-worn device wearer. The ear-worn device wearer evaluation 1112 can also include receiving care provider input 1110, such as input from a physician, an audiologist, a physical therapist, or the like. In various embodiments, the vestibular rehabilitation management system 100 can be configured to evaluate the wearer of the ear-worn device 102 by matching demographic and/or medically relevant data such as at least one of their age, gender, sex at birth, size or medical record with records of other patients stored in a database (stored in compliance with all applicable regulations) that includes information regarding exercise performed by the same. By way of example, if the database stores records including that most 75-year-old females with an artificial hip tend to plateau at a difficulty level of “X”, then the system can suggest an initial difficulty level below “X” and ramp up the difficulty over time to achieve “X”.

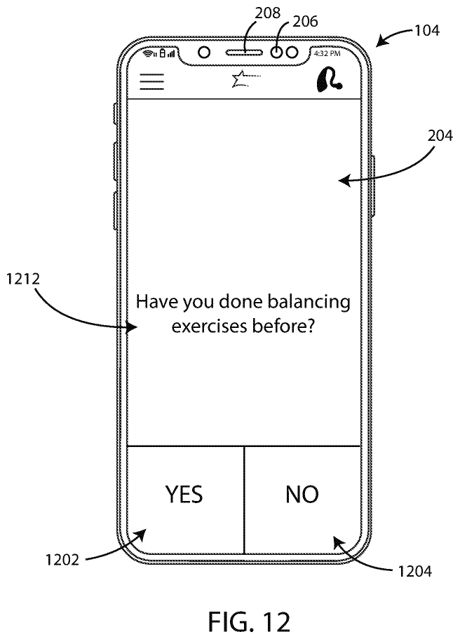

As referenced above, the system can pose questions of a device wearer (and/or a third party) as part of the evaluation of a device wearer. Referring now to FIG. 12, a schematic view of an accessory device 104 is shown in accordance with various embodiments herein. The accessory device 104 can present a query 1212 on the visual display 204 thereof. In some embodiments, the accessory device 104 can include a speaker 208 and also present a query audibly. The accessory device 104 also includes a camera 206, a microphone, a first user input button 1202 and a second user input button 1204. One or more of the camera 206, a microphone, a first user input button 1202 and a second user input button 1204 can be used to receive input from the device wearer and/or a third party in response to the query 1212.

In various embodiments, the system can evaluate movement of the device wearer as part of the evaluation of the device wearer. In various embodiments, wherein movement can be analyzed to detect at least one of swaying, stumbles, and falls.

In various embodiments, the vestibular rehabilitation management system can be configured to evaluate the wearer of the ear-worn device by evaluating maximum degree of lean before imbalance occurs, i.e., the user's limit of stability. In various embodiments, the vestibular rehabilitation management system can be configured to evaluate the wearer of the ear-worn device by evaluating recovery movements after imbalance occurs (either actively induced or passively sensed).

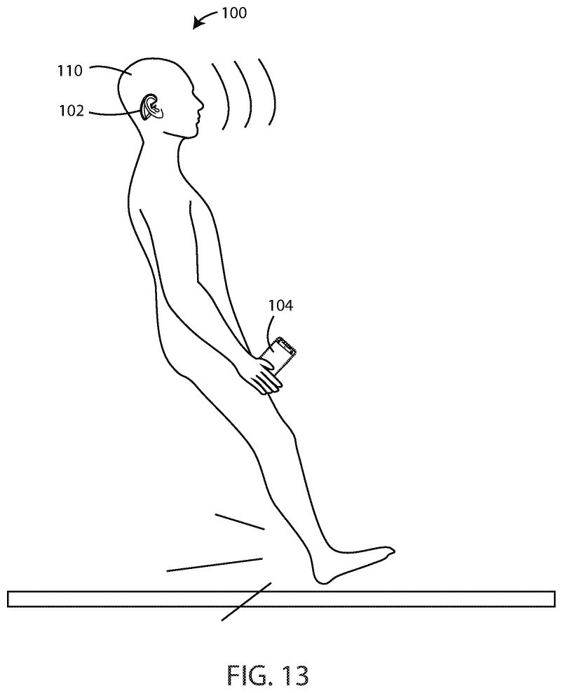

Referring now to FIG. 13, a schematic view of a device wearer 110 and components of a vestibular rehabilitation management system 100 are shown in accordance with various embodiments herein. As before, the vestibular rehabilitation management system 100 includes an ear-worn device 102. The vestibular rehabilitation management system 100 also includes or interfaces with an accessory device 104.

In various embodiments, the vestibular rehabilitation management system 100 can be configured to evaluate the wearer of the ear-worn device 102 by measuring a reaction speed of the wearer of the ear-worn device 102. Aspects of measuring reaction speed are described in U.S. Pat. Appl. No. 62/876,458 titled “Ear-Worn Device Based Measurement of Reaction or Reflex Speed”, the content of which is herein incorporated by reference in its entirety.

In various embodiments, the vestibular rehabilitation management system 100 can be configured to evaluate the wearer of the ear-worn device 102 by evaluating imbalance recovery. In various embodiments, the vestibular rehabilitation management system 100 can be configured to evaluate the wearer of the ear-worn device 102 by evaluating imbalance recovery speed.

In various embodiments, wherein evaluating imbalance recovery includes measuring a degree of head or body sway after imbalance. Generally, the longer it takes for head or body sway to reach a normal value for the individual the worse their balance function is. In various embodiments, wherein evaluating imbalance recovery includes measuring foot impact magnitude after imbalance. All things being equal, a larger foot impact may indicate that the device wearer was more off-balance and thus may have worse balance function. In various embodiments, wherein evaluating imbalance recovery includes measuring maximum foot speed after imbalance.

Imbalance recovery evaluation can be either active or passive. In various embodiments, the vestibular rehabilitation management system 100 can be configured to evaluate the wearer of the ear-worn device 102 by actively initiating imbalance and then evaluating imbalance recovery. Imbalance may be initiated through various techniques including caloric and/or electrical stimulation of the inner ear. However, in various embodiments, the vestibular rehabilitation management system 100 can be configured to evaluate the wearer of the ear-worn device 102 by passively detecting imbalance (e.g., waiting for a circumstance of imbalance to occur naturally) and then evaluating imbalance recovery after the same.

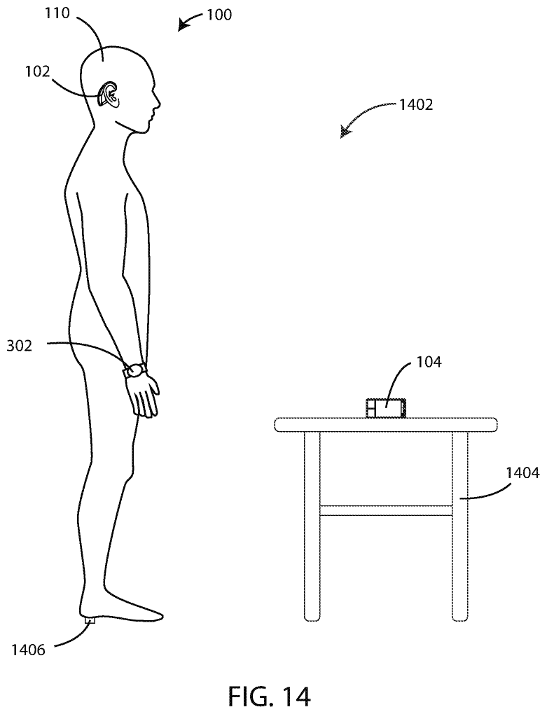

It will be appreciated that useful information regarding the device-wearer's exercise progress can be drawn by evaluating movement data and, in some cases, comparing movement data gathered by one device with movement data gathered by another device. In some cases, even a lack of movement can be probative. For example, if the system detects that the accessory device is still in communication but is suddenly indicating no movement, then this can be consistent with the device wearer setting the accessory device down, which may be necessary or preferred for some exercises. Referring now to FIG. 14, a schematic view of a device wearer 110 and components of a vestibular rehabilitation management system 100 are shown in accordance with various embodiments herein. The vestibular rehabilitation management system 100 includes an ear-worn device 102. The vestibular rehabilitation management system 100 also includes and/or interfaces with an accessory device 104. The vestibular rehabilitation management system 100 can also include and/or interface with an additional device 302. In some cases, the vestibular rehabilitation management system 100 can also include and/or interface with an accessory sensing device 1406. In some embodiments, the accessory sensing device 1406 could be a balance board or a treadmill. In some embodiments, the balance board or treadmill can include an active component such as a stimulator or an actuator to either confuse one aspect of the balance system or to perturb the balance of the user.

In this example, the environment 1402 of the device wearer 110 can include a piece of furniture 1404. If the accessory device is set on the floor or on the furniture 1404, then the movement data gathered from a movement sensor associated with the accessory device 104 can indicate that movement has stopped. Detecting the cessation of movement can be useful for various purposes. In some embodiments, it can be used to detect when the device wearer is taking a break. In some embodiments, such as with an exercise that involves both hands, it can be used to detect when the device wearer is performing the exercise.

Thus, in various embodiments, movement data below a threshold value can be indicative of the accessory device 104 not being held. In various embodiments, the vestibular rehabilitation management system 100 can be configured to record patterns of the accessory device 104 being held and not being held. In various embodiments, the vestibular rehabilitation management system 100 can be configured to pace exercises based on patterns of the accessory device 104 being held and not being held.

In various embodiments, the environment 1402 of the wearer of the ear-worn device 102 can be evaluated. For some types of exercises, the environment 1402 of the ear-worn device wearer needs to be relatively open so they have sufficient space to perform the exercise. In various embodiments, the vestibular rehabilitation management system 100 can be configured to receive information regarding the environment 1402 of the wearer of the ear-worn device 102 from an accessory device 104. In various embodiments, the information regarding the environment 1402 of the wearer can include information regarding obstacles in the environment 1402 of the wearer of the ear-worn device 102. Obstacles in the environment of the wearer can include, for example, furniture 1404, rugs, power cords, pets, other persons, etc. Each exercise stored by the system can include one or more parameters indicative of how much space is reasonably required to perform the exercise. If the environment 1402 lacks sufficient space for the particular exercise as indicated by the one or more parameters, then that exercise will not be suggested to the ear-worn device wearer. In some embodiments, the system may evaluate the environment 1402 to determine if it lacks sufficient space for the particular exercise based on the presence of one or more obstacles in the environment. In some embodiments, the system may incorporate one or more of instructing the user to navigate about the obstacles in the environment and evaluating the user's performance when navigating about the obstacles in the environment. It is known in the art that navigating complex environments increases the cognitive load on the user performing e.g., a walking task.

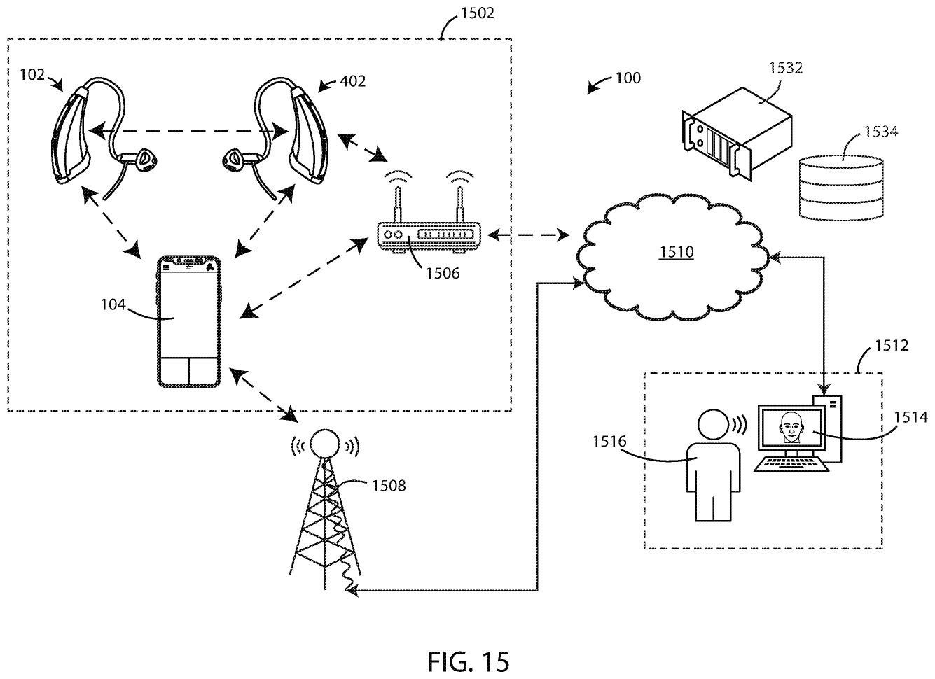

In various embodiments herein, data/signals/information can be conveyed to one or more remote locations for processing and/or for interface with a third party such as a care provider. Further, data/signals/information can be sent from a remote location to one or more of an ear-worn device and/or an accessory device. Referring now to FIG. 15, a schematic view of a vestibular rehabilitation management system 100 is shown in accordance with various embodiments herein. FIG. 15 shows a first ear-worn device 102, a second ear-worn device 402, and an accessory device 104 that are all at a first location 1502. The first location 1502 can be the current environment of the ear-worn device wearer. FIG. 15 also shows various data communication equipment such as a router 1506 and a cellular communications tower 1508, which can be used to convey data/signals to or from parts of the system. The data communication equipment can be used to provide a connection to the cloud 1510 and/or one or more remote servers 1532 (real or virtual and may be part of the cloud) and databases 1534 (with may be part of the cloud). In some embodiments, connections to the cloud and/or remote servers can be used to offload certain processing tasks, such as computing related to evaluating the device wearer and/or selecting exercises for the same. In some embodiments, data used to evaluate the device wearer and/or select exercises can be stored in the cloud 1510 and/or on remote servers 1532.

In various embodiments, the cloud 1510 and/or data communication equipment can be used to provide a connection with a third party 1516 and a remote location 1512. The third party 1516 can receive information through a computing device 1514 at the remote location. In various embodiments, the vestibular rehabilitation management system 100 can be further configured to present the selected vestibular training exercise to the third party 1516 (which could be a care provider at the remote location 1512 or not) for the wearer of the ear-worn device 102 as a suggestion.

In various embodiments, a vestibular rehabilitation management system 100 herein can be configured to initiate or detect performance of an exercise by a wearer of the ear-worn device 102, monitor signals from the sensor package to detect a performance status of an exercise, and send information regarding the exercise to a remote location. In various embodiments, the vestibular rehabilitation management system 100 can be further configured to initiate a real-time connection with a third party 1516.

In various embodiments, the vestibular rehabilitation management system 100 can be further configured to receive input from a third party 1516 regarding the exercise or a future exercise. In various embodiments, the vestibular rehabilitation management system 100 sends information regarding the exercise to the remote location 1512 if the performance status represents a change over a previous performance status crossing a threshold value.

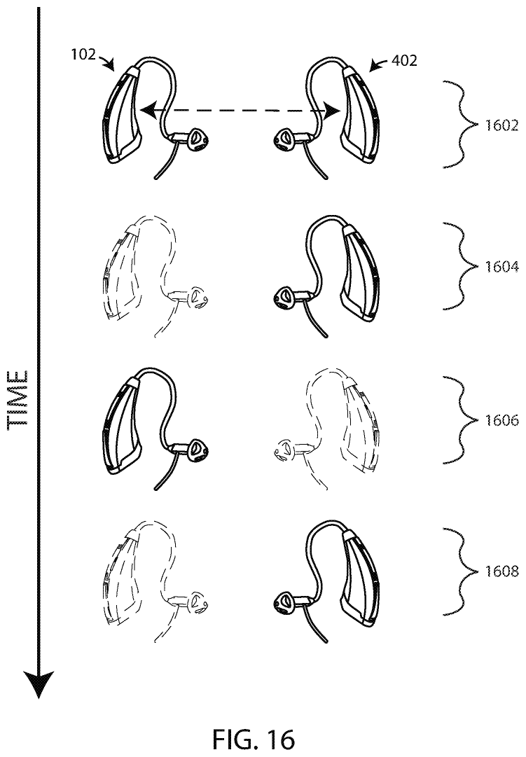

Some types of sensors (such as gyroscopes) can use significant amounts of energy during operation. Further some types of system operations such as wireless radio operation can use significant amounts of energy. This is significant because in many scenarios an ear-worn device will be operating off of power provided by a battery with finite energy storage capability. In some embodiments where there are two ear-worn devices, the devices can work together and conserve energy by effectively sharing the use of a single sensor by sequentially using a sensor associated with one device and then using a sensor associated with the other device. Similarly, in some embodiments where there are two ear-worn devices, the devices can work together and conserve energy by effectively having only one device execute wireless radio operation at given points in time so as to eliminate or reduce redundancy and save substantial energy. Referring now to FIG. 16, a schematic diagram of a pair of ear-worn devices (102, 402) trading off in sensor operation and/or wireless radio operation is shown in accordance with various embodiments herein. Time periods of operation can include a first time period 1602 in which sensors (or at least a particular type of sensor) is active in both ear-worn devices and/or wireless radio operations are being conducted by both ear-worn devices. Then, in a second time period 1604, the sensor(s) and/or wireless radio operations of the first ear-worn device 102 are turned off and the sensor(s) from the second ear-worn device 402 gather data and/or the second ear-worn device 402 executes wireless radio operations. Then, in a third time period 1606, the sensor(s) and/or wireless radio operations of the second ear-worn device 402 are turned off and the sensor(s) from the first ear-worn device 102 are turned on and gather data and/or the first ear-worn device 102 executes wireless radio operations. Then, in a fourth time period 1608, the sensor(s) and/or wireless radio operations of the first ear-worn device 102 are turned off and the sensor(s) from the second ear-worn device 402 are turned on and gather data and/or the second ear-worn device 402 executes wireless radio operations. The specific patterns of switching between the two devices can vary. However, the key is that energy can be saved by relying upon only one or more sensors from a specific device at various points in time (e.g., by at least temporarily eliminating sensor and/or wireless radio redundancy). It will be appreciated that embodiments herein include applying the same technique (eliminating or reducing redundancy to save energy) to any other operation which is conducted by the ear-worn devices that would otherwise be conducted redundantly and consumes significant energy.

As such, in various embodiments a vestibular rehabilitation management system 100 can include a first ear-worn device 102 and a second ear-worn device 402 wherein the vestibular rehabilitation management system 100 can be configured to initiate or detect initiation of an exercise, turn on a first motion sensor associated with the first ear-worn device 102 and monitor a signal from the first motion sensor, turn off the first motion sensor, turn on the second motion sensor associated with the second ear-worn device 402, and monitor a signal from the second motion sensor.

In various embodiments, the vestibular rehabilitation management system 100 can be configured to turn off the first motion sensor, turn on the second motion sensor, and monitor a signal from the second motion sensor based on a time schedule. In various embodiments, the vestibular rehabilitation management system 100 can be configured to turn off the first motion sensor, turn on the second motion sensor, and monitor a signal from the second motion sensor based on a comparison of a remaining stored charge of the first battery versus a remaining stored charge of the second battery. In various embodiments, the first motion sensor and the second motion sensor can include a gyroscope.

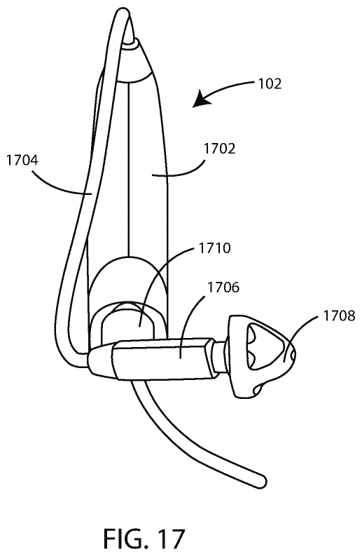

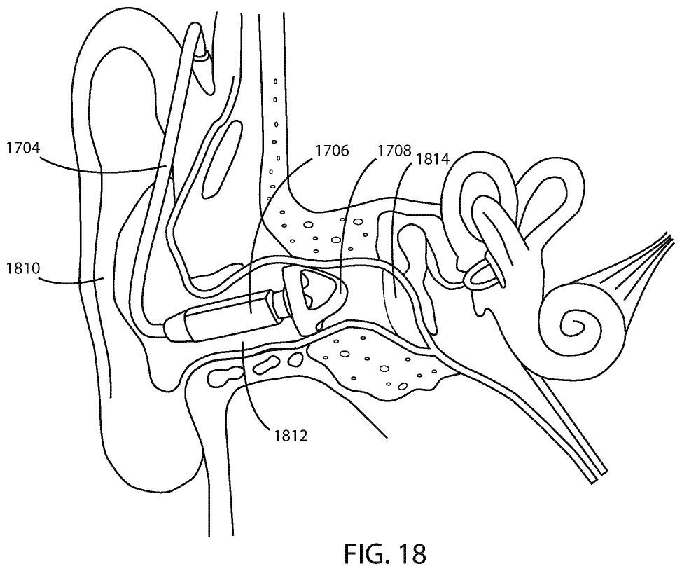

Referring now to FIG. 17, a schematic view of an ear-worn device 102 is shown in accordance with various embodiments herein. The ear-worn device 102 can include a hearing device housing 1702. The hearing device housing 1702 can define a battery compartment 1710 into which a battery can be disposed to provide power to the device. The ear-worn device 102 can also include a receiver 1706 adjacent to an earbud 1708. The receiver 1706 an include a component that converts electrical impulses into sound, such as an electroacoustic transducer, speaker, or loud speaker. Such components can be used to generate an audible stimulus in various embodiments herein. A cable 1704 or connecting wire can include one or more electrical conductors and provide electrical communication between components inside of the hearing device housing 1702 and components inside of the receiver 1706.

The ear-worn device 102 shown in FIG. 17 is a receiver-in-canal type device and thus the receiver is designed to be placed within the ear canal. However, it will be appreciated that may different form factors for ear-worn devices are contemplated herein. As such, ear-worn devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices.

The term “ear-worn device” shall also refer to devices that can produce optimized or processed sound for persons with normal hearing. Ear-worn devices herein can include hearing assistance devices. In some embodiments, the ear-worn device can be a hearing aid falling under 21 C.F.R. § 801.420. In another example, the ear-worn device can include one or more Personal Sound Amplification Products (PSAPs). In another example, the ear-worn device can include one or more cochlear implants, cochlear implant magnets, cochlear implant transducers, and cochlear implant processors. In another example, the ear-worn device can include one or more “hearable” devices that provide various types of functionality. In other examples, ear-worn devices can include other types of devices that are wearable in, on, or in the vicinity of the user's ears. In other examples, ear-worn devices can include other types of devices that are implanted or otherwise osseointegrated with the user's skull; wherein the device is able to facilitate stimulation of the wearer's ears via the bone conduction pathway.

Ear-worn devices of the present disclosure can incorporate an antenna arrangement coupled to a radio, such as a 2.4 GHz radio. The radio can conform to an IEEE 802.11 (e.g., WIFI) or BLUETOOTH® (e.g., BLE, BLUETOOTH® 4.2 or 5.0) specification, for example. It is understood that ear-worn devices of the present disclosure can employ other radios, such as a 900 MHz radio. Ear-worn devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files.

As mentioned above, the ear-worn device 102 shown in FIG. 17 can be a receiver-in-canal type device and thus the receiver is designed to be placed within the ear canal. Referring now to FIG. 18, a schematic view is shown of an ear-worn device 102 disposed within the ear of a subject in accordance with various embodiments herein. In this view, the receiver 1706 and the earbud 1708 are both within the ear canal 1812, but do not directly contact the tympanic membrane 1814. The hearing device housing is mostly obscured in this view behind the pinna 1810, but it can be seen that the cable 1704 passes over the top of the pinna 1810 and down to the entrance to the ear canal 1812.

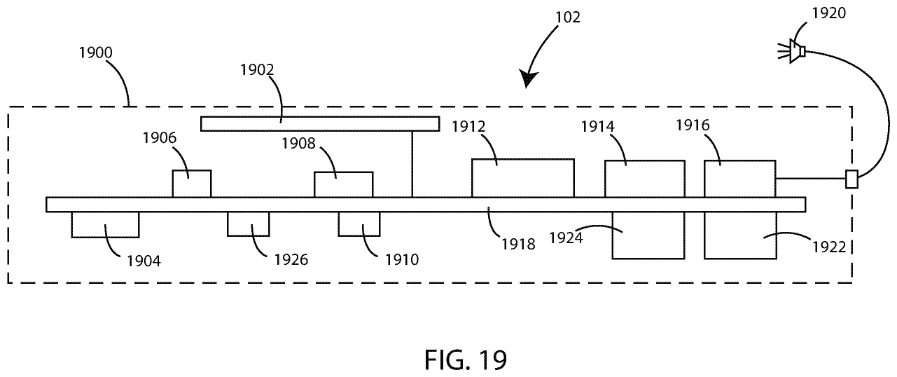

Referring now to FIG. 19, a schematic block diagram of components of an ear-worn device is shown in accordance with various embodiments herein. The block diagram of FIG. 19 represents a generic ear-worn device for purposes of illustration. The ear-worn device 102 shown in FIG. 19 includes several components electrically connected to a flexible mother circuit 1918 (e.g., flexible mother board) which is disposed within housing 1900. A power supply circuit 1904 can include a battery and can be electrically connected to the flexible mother circuit 1918 and provides power to the various components of the ear-worn device 102. One or more microphones 1906 are electrically connected to the flexible mother circuit 1918, which provides electrical communication between the microphones 1906 and a digital signal processor (DSP) 1912. Among other components, the DSP 1912 incorporates or is coupled to audio signal processing circuitry configured to implement various functions described herein. A sensor package 1914 can be coupled to the DSP 1912 via the flexible mother circuit 1918. The sensor package 1914 can include one or more different specific types of sensors such as those described in greater detail below. One or more user switches 1910 (e.g., on/off, volume, mic directional settings) are electrically coupled to the DSP 1912 via the flexible mother circuit 1918.

An audio output device 1916 is electrically connected to the DSP 1912 via the flexible mother circuit 1918. In some embodiments, the audio output device 1916 comprises an electroacoustic transducer or speaker (coupled to an amplifier). In other embodiments, the audio output device 1916 comprises an amplifier coupled to an external receiver 1920 adapted for positioning within an ear of a wearer. The external receiver 1920 can include an electroacoustic transducer, speaker, or loud speaker. The ear-worn device 102 may incorporate a communication device 1908 coupled to the flexible mother circuit 1918 and to an antenna 1902 directly or indirectly via the flexible mother circuit 1918. The communication device 1908 can execute wireless radio operations. The communication device 1908 can be a BLUETOOTH® transceiver, such as a BLE (BLUETOOTH® low energy) transceiver or other transceiver(s) (e.g., an IEEE 802.11 compliant device). The communication device 1908 can be configured to communicate with one or more external devices, such as those discussed previously, in accordance with various embodiments. In various embodiments, the communication device 1908 can be configured to communicate with an external visual display device such as a smart phone, a video display screen, a tablet, a computer, smart glasses, a smart mirror, a virtual reality or augmented reality display, a hologram, or the like.

In various embodiments, the ear-worn device 102 can also include a control circuit 1922 and a memory storage device 1924. The control circuit 1922 can be in electrical communication with other components of the device. In some embodiments, a clock circuit 1926 can be in electrical communication with the control circuit. The control circuit 1922 can execute various operations, such as those described herein. The control circuit 1922 can include various components including, but not limited to, a microprocessor, a microcontroller, an FPGA (field-programmable gate array) processing device, an ASIC (application specific integrated circuit), or the like. The memory storage device 1924 can include both volatile and non-volatile memory. The memory storage device 1924 can include ROM, RAM, flash memory, EEPROM, SSD devices, NAND chips, and the like. The memory storage device 1924 can be used to store data from sensors as described herein and/or processed data generated using data from sensors as described herein.

It will be appreciated that various of the components described in FIG. 19 can be associated with separate devices and/or accessory devices to the ear-worn device. By way of example, microphones can be associated with separate devices and/or accessory devices. Similarly, audio output devices can be associated with separate devices and/or accessory devices to the ear-worn device.

Mobile electronic devices herein can include various different components. In some embodiments, the mobile electronic device can be a personal communications device, such as a smart phone. However, the mobile electronic device can also be other things such as a wearable device, a handheld computing device, a dedicated location determining device (such as a handheld GPS unit), or the like.

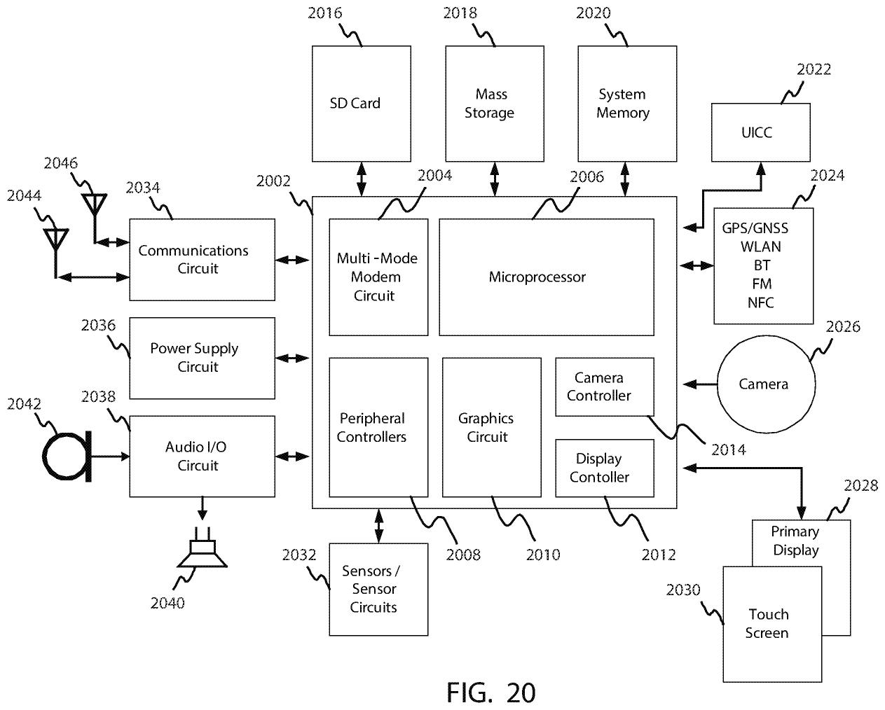

Referring now to FIG. 20, a schematic block diagram is shown of components of an accessory device (which could be a personal communications device, smart phone, or another type of mobile electronic device) in accordance with various embodiments herein. This block diagram is just provided by way of illustration and it will be appreciated that mobile electronic devices can include greater or lesser numbers of components. The mobile electronic device in this example can include a control circuit 2002. The control circuit 2002 can include various components which may or may not be integrated. In various embodiments, the control circuit 2002 can include a microprocessor 2006, which could also be a microcontroller, FPGA, ASIC, or the like. The control circuit 2002 can also include a multi-mode modem circuit 2004 which can provide communications capability via various wired and wireless standards. The control circuit 2002 can include various peripheral controllers 2008. The control circuit 2002 can also include various sensors/sensor circuits 2032. The control circuit 2002 can also include a graphics circuit 2010, a camera controller 2014, and a display controller 2012. In various embodiments, the control circuit 2002 can interface with an SD card 2016, mass storage 2018, and system memory 2020. In various embodiments, the control circuit 2002 can interface with universal integrated circuit card (UICC) 2022. A spatial location determining circuit can be included and can take the form of an integrated circuit 2024 that can include components for receiving signals from GPS, GLONASS, BeiDou, Galileo, SBAS, WLAN, BT, FM, and NFC type protocols. In various embodiments, the mobile electronic device can include a camera 2026. In various embodiments, the control circuit 2002 can interface with a primary display 2028 that can also include a touch screen 2030 or another suitable user control interface. In various embodiments, an audio I/O circuit 2038 can interface with the control circuit 2002 as well as a microphone 2042 and a speaker 2040. In various embodiments, a power supply circuit 2036 can interface with the control circuit 2002 and/or various other circuits herein in order to provide power to the system. In various embodiments, a communications circuit 2034 can be in communication with the control circuit 2002 as well as one or more antennas (2044, 2046).

### Methods

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method of monitoring or managing vestibular rehabilitation exercise is included, the method including evaluating a wearer of an ear-worn device and selecting a vestibular training exercise based on the evaluation.

In an embodiment, a method of monitoring or managing vestibular rehabilitation exercise is included, the method including monitoring signals from a motion sensor to detect system declared performance of an exercise, monitoring signals from a microphone to detect wearer declared performance of an exercise, and comparing system declared performance of the exercise with wearer declared performance of the exercise.

In an embodiment, a method of monitoring or managing vestibular rehabilitation exercise is included, the method including evaluating a wearer of an ear-worn device by monitoring signals from a microphone to detect a voice of a wearer of the ear-worn device and selecting a vestibular training exercise based on the evaluation.

In an embodiment, a method of monitoring or managing vestibular rehabilitation exercise is included, the method including initiating or detecting performance of an exercise by a wearer of the ear-worn device, monitoring signals from the sensor package to detect a performance status of an exercise, and sending information regarding the exercise to a remote location.

In an embodiment, a method of monitoring or managing vestibular rehabilitation exercise is included, the method including initiating or detecting initiation of an exercise, turning on a first motion sensor and monitoring a signal from the first motion sensor, turning off the first motion sensor, turning on a second motion sensor, and monitoring a signal from the second motion sensor.

In an embodiments, a method of monitoring or managing vestibular rehabilitation exercise is included, the method including initiating or detecting initiation of an exercise, selecting either a first ear-worn device or a second ear-worn device, turning on the motion sensor of the selected device and/or executing wireless radio operations with the selected device, turning off the motion sensor of the non-selected device and/or ceasing or reducing wireless radio operations of the non-selected device.

### Sensors

Ear-worn devices herein can include one or more sensor packages (including one or more discrete or integrated sensors) to provide data. The sensor package can comprise one or a multiplicity of sensors. In some embodiments, the sensor packages can include one or more motion sensors amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GME, etc.) sensors may be used to detect motion or changes in position. In some embodiments, biometric sensors may be used to detect body motions or physical activity. Motion sensors can be used to track movement of a patient in accordance with various embodiments herein.

In some embodiments, the motion sensor(s) can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of a motion sensor, (e.g., IMU, and accelerometer (3, 6, or 9 axis), a gyroscope, a barometer, an altimeter, a magnetometer, a magnetic sensor, an eye movement sensor, a pressure sensor), an acoustic sensor, a telecoil, a heart rate sensor, a global positioning system (GPS), a barometer, a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter, a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

In some embodiments, the sensor package can be part of an ear-worn device. However, in some embodiments, the sensor packages can include one or more additional sensors that are external to an ear-worn device. For example, various of the sensors described above can be part of a wrist-worn or ankle-worn sensor package, or a sensor package supported by a chest strap.

Data produced by the sensor(s) of the sensor package can be operated on by a processor or control circuit of the device or system.

As used herein the term “inertial measurement unit” or “IMU” shall refer to an electronic device that can generate signals related to a body's specific force and/or angular rate. IMUs herein can include one or more accelerometers and gyroscopes (3, 6, or 9 axis) to detect linear acceleration and a gyroscope to detect rotational rate. In some embodiments, an IMU can also include a magnetometer to detect a magnetic field.

The eye movement sensor may be, for example, an electrooculographic (EOG) sensor, such as an EOG sensor disclosed in commonly owned U.S. Pat. No. 9,167,356, which is incorporated herein by reference. The pressure sensor can be, for example, a MEMS-based pressure sensor, a piezo-resistive pressure sensor, a flexion sensor, a strain sensor, a diaphragm-type sensor and the like.

The temperature sensor can be, for example, a thermistor (thermally sensitive resistor), a resistance temperature detector, a thermocouple, a semiconductor-based sensor, an infrared sensor, or the like.

The blood pressure sensor can be, for example, a pressure sensor. The heart rate sensor can be, for example, an electrical signal sensor, an acoustic sensor, a pressure sensor, an infrared sensor, an optical sensor, or the like.

The oxygen saturation sensor (such as a blood oximetry sensor) can be, for example, an optical sensor, an infrared sensor, or the like.

The electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-worn device. In addition to the external sensors discussed hereinabove, the sensor package can comprise a network of body sensors (such as those listed above) that sense movement of a multiplicity of body parts (e.g., arms, legs, torso). In some embodiments, the ear-worn device can be in electronic communication with the sensors or processor of a medical device (implantable, wearable, external, etc.).

### Exercise Selection

Various embodiments herein include an exercise selection. Further details about the exercise selection are described above and provided as follows. However, it will be appreciated that this is merely provided by way of example and that further variations are contemplated herein.

In various embodiments, the vestibular training exercise selection includes selection of a dominant leg. In various embodiments, the vestibular training exercise selection includes selection of a vestibular training exercise difficulty. In various embodiments, the vestibular training exercise difficulty is selected with respect to a least one of a strength intensity index value, a vestibular function intensity index value, postural stability intensity index value, a reaction speed intensity index value, a stamina intensity index value, a speed intensity index value, a visual intensity index value, an environmental stimulation intensity index value, a proprioception intensity index value, and a cognitive load intensity index value. Index values are not particularly limited, but in some embodiments can range from 1 to 5, 1 to 10, or from 1 to 100. Exercises stored by the system can each have an assigned index value for overall difficulty and/or for each component of difficult such as strength, vestibular function, and reaction speed. In various embodiments, the vestibular training exercise difficulty is selected with respect to exercise repetitions and/or duration. For example, standing on one leg for 60 seconds is of greater difficulty than standing on one leg for 30 seconds.

In various embodiments, the vestibular rehabilitation management system is configured to select a vestibular training exercise based on a mode setting stored or accessed by the vestibular rehabilitation management system. In various embodiments, the mode setting includes at least one of a training mode, a maintenance mode, and a daily life mode. In a training mode the series of exercises presented to the device wearer can ramp up in difficulty relatively quickly so as to maximize improvements obtained through vestibular rehabilitation exercises. In a maintenance mode the series of exercises presented to the device wearer can ramp up more gradually or not at all in view of a device wearer that may simply be looking to maintain their current degree of balance system function. In a daily life mode the series of exercises can be tailored to and fit into the lifestyle of the device wearer. For example, if the system detects substantial movement throughout the day or otherwise detects that the device wearer is exhausted then the system may not suggest any exercises to the device wearer or may suggest only those with relatively low difficulty.

It can be advantageous to not push device wearers beyond their capabilities. In various embodiments, no vestibular training exercise is selected (e.g., vestibular training can be skipped) if detected movement crosses a threshold value. In this manner, if the device wearer has already had a lot of physical activity, the vestibular training can be skipped.

In various embodiments, the vestibular rehabilitation management system is further configured to present the selected vestibular training exercise to the wearer of the ear-worn device as a suggestion. In various embodiments, the vestibular rehabilitation management system can select a time to present the selected vestibular training exercise to the wearer of the ear-worn device as a suggestion. In various embodiments, the time can be based on preferences of the wearer of the ear-worn device as previously input into the system. For example, the wearer can input into or otherwise indicate to the system that they prefer to exercise between the hours of 7 and 9 AM. In various embodiments, the time can be based on detected movement patterns of the wearer of the ear-worn device. For example, when the vestibular rehabilitation management system detects a period of relatively little movement (such as by detecting that motions sensors indicate movement (or an averaged movement value) crossing below a threshold value then the system can suggest an exercise to the wearer of the ear-worn device.

In various embodiments, the vestibular rehabilitation management system is further configured to present the selected vestibular training exercise to a care provider for the wearer of the ear-worn device as a suggestion.

Exemplary exercises can be stored by one or more components of the system (including instructions as well as parameters regarding difficulty, space required, etc.). Exemplary exercises herein can include exercises involving standing, standing with feet in particular placements (feet together, feet in a “T”, etc.), standing on one leg, hopping on one leg, rotating the head side to side, rotating the head up and down (like exaggerated nodding of the head), leaning to one side or another, leaning forward or back, focusing the eyes on a given point in space while moving the body and/or the head, walking in a straight line, walking in other than a straight line, turning the body, squatting, and the like.

### Wearer Evaluation

Various embodiments herein include a wearer evaluation. Further details about the wearer evaluation are provided as follows. However, it will be appreciated that this is merely provided by way of example and that further variations are contemplated herein.

In various embodiments, the vestibular rehabilitation management system is configured to evaluate the wearer of the ear-worn device by sensing their movement over a time period preceding selection of the vestibular training exercise. The time period can vary. In various embodiments, the time period preceding selection of the vestibular training exercise spans about 30 seconds to about 12 weeks, or about 1 minute to 4 weeks, or about 1 minute to 60 minutes. In various embodiments, the time period preceding selection of the vestibular training exercise includes a time period wherein the wearer of the ear-worn device is performing an exercise (which could be a vestibular training exercise or another type of training exercise).

In various embodiments, the vestibular rehabilitation management system is configured to evaluate the wearer of the ear-worn device by sensing their ability to complete a preceding exercise (e.g., did detected motion cross a threshold value for a particular exercise, did detected rotation cross a threshold value for a particular exercise, etc.). In various embodiments, the vestibular rehabilitation management system is configured to evaluate the wearer of the ear-worn device by sensing an accuracy of completion of a preceding exercise. In various embodiments, the vestibular rehabilitation management system is configured to evaluate the wearer of the ear-worn device by determining the wearer's performance improvement over a series of preceding exercises.

In various embodiments, the vestibular rehabilitation management system is configured to evaluate the wearer of the ear-worn device by evaluating a health record input. In various embodiments, the vestibular rehabilitation management system is configured to evaluate the wearer of the ear-worn device by evaluating consumption (or administration) of a medication. For example, certain medications may make a device wearer temporarily unsteady. It can be useful to consider medication-induced unsteadiness (or other medication induced effects) as part of the evaluation of the patient. In some cases, medication administration can be detected directly or indirectly. For example, if a particular medication is prescribed to be taken three times a day with food, then using signals from a microphone and/or from a motion sensor can be analyzed to detect patterns consistent with eating food (such as a pattern consistent with mastication) and it can be inferred that a medication administration event has taken place at the same time. Aspects of medication administration detection and tracking are described in U.S. Pat. Appl. No. 62/810,684, entitled “System and Method for Managing Pharmacological Therapeutics Including a Health Monitoring Device”, the content of which is herein incorporated by reference.

In various embodiments, the vestibular rehabilitation management system is configured to evaluate the wearer of the ear-worn device by matching at least one of their age, gender, sex at birth, size or medical record specifics with records stored in a database representing a population of patients. Matching can be performed using various pattern matching algorithms and/or can be conducted using a machine learning approach. In some cases, matching can lead to identification of similar patients/populations and information regarding desirable exercise difficulty and/or types of exercise can be stored along with the patient/population data. As such, this type of matching analysis can directly lead to a suggestion for exercise for the device wearer.

In various embodiments, the vestibular rehabilitation management system is configured to evaluate the wearer of the ear-worn device by evaluating answers to one or more questions posed to the ear-worn device wearer. In various embodiments, the vestibular rehabilitation management system is configured to evaluate the wearer of the ear-worn device by measuring a degree of head or body sway.

In various embodiments, the vestibular rehabilitation management system is configured to evaluate the wearer of the ear-worn device by evaluating maximum lean before imbalance occurs.

In various embodiments, the vestibular rehabilitation management system is configured to evaluate the wearer of the ear-worn device by measuring a reaction speed of the wearer of the ear-worn device. Aspects of measuring reaction speed are described in U.S. Pat. Appl. No. 62/876,458 titled “Ear-Worn Device Based Measurement of Reaction or Reflex Speed”, the content of which is herein incorporated by reference in its entirety.

In various embodiments, the vestibular rehabilitation management system is configured to analyze movement to detect at least one of swaying, stumbles, and falls. Exemplary aspects of fall detection can be found in U.S. patent application Ser. No. 16/714,339, entitled “Hearing Assistance System with Enhanced Fall Detection Features”, the content of which is herein incorporated by reference.

In various embodiments, the vestibular rehabilitation management system is configured to evaluate the wearer of the ear-worn device by evaluating imbalance recovery. In various embodiments, the vestibular rehabilitation management system is configured to evaluate the wearer of the ear-worn device by evaluating imbalance recovery speed. For example, how long does it take for data from motion sensors to return to normal levels for the device wearer after a circumstance of imbalance has occurred.

In various embodiments, wherein evaluating imbalance recovery includes measuring a degree of head or body sway after imbalance. In various embodiments, wherein evaluating imbalance recovery includes measuring foot impact magnitude after imbalance. In various embodiments, wherein evaluating imbalance recovery includes measuring foot speed after imbalance.

In various embodiments, the vestibular rehabilitation management system is configured to evaluate the wearer of the ear-worn device by actively initiating imbalance and then evaluating imbalance recovery. In various embodiments, the vestibular rehabilitation management system is configured to evaluate the wearer of the ear-worn device by passively detecting imbalance and then evaluating imbalance recovery.

### System Detected Performance

Various embodiments herein include vestibular rehabilitation management system detected performance. Further details about the system detected performance are provided as follows. However, it will be appreciated that this is merely provided by way of example and that further variations are contemplated herein.

In various embodiments, a vestibular rehabilitation management system can include an ear-worn device including a control circuit, a microphone in communication with the control circuit, and a motion sensor in communication with the control circuit. The ear-worn device can be configured to monitor signals from the motion sensor to detect system declared performance of an exercise, monitor signals from the microphone to detect wearer declared performance of an exercise, and compare system declared performance of the exercise with wearer declared performance of the exercise.

Wearer declared performance of an exercise can be detected by the system in various ways. In some embodiments, the microphone can pick up sound that is then processed to recognize counts (“1, 2, 3, . . . ”) spoken by the device wearer. In some embodiments, the device wearer interface with one or more devices of the system such as by pressing input buttons on a display screen of an accessory device. System declared performance of an exercise can be detected by the system in various ways, depending on the particular exercise in question. By way of example, for an exercise that includes turning the head from side to side a signal from a motion sensor can be evaluated in order to detect head rotation crossing a threshold magnitude. For an exercise that includes standing up and sitting down, a signal from a motion sensor can be evaluated in order to detect vertical movement of a magnitude consistent with standing up and sitting down. For an exercise that includes standing up and sitting down, a signal from a blood pressure sensor and/or a heart rate sensor can be evaluated in order to detect characteristic changes in blood pressure and/or heart rate associated with standing up and sitting down. For an exercise that includes turning the body, a signal from a motion sensor can be evaluated in order to detect rotational movement crossing a threshold value.

In various embodiments, the vestibular rehabilitation management system configured to initiate a report to a third party if the system declared performance of the exercise and the wearer declared performance of the exercise deviates by at least a threshold value in terms of number of exercises and/or exercise repetitions performed or another measure of exercise performance. In some cases, differences between system declared and wearer declared performance can be due to sensors not being properly calibrated. In various embodiments, the vestibular rehabilitation management system configured to initiate a calibration process if the system declared performance of the exercise and the wearer declared performance of the exercise deviates by at least a threshold value. For example, the wearer can be instructed to perform a particular exercise (with or without supervision of a care provider) and the system can record all movement with motion sensors. The motion sensor signals can be analyzed to identify a signature of the exercise (degree of motion, degree of rotation, etc.) which can then be stored in memory and later used to identify particular exercises being performed and/or repetitions of the same.

It will be appreciated that various types of exercise herein may include an element of directionality. For example, exercises can include a head turn, a body turn, and the like. The starting position that a wearer of the device may be in (at the start of an exercise session, at the start of a particular exercise movement, etc.) can have relevance for various purposes including providing appropriate guidance as well as detecting performance. In some embodiments herein, the system can be configured to include auto-centering logic/functionality to establish an appropriate reference point (head position, body position, head position relative to body position, etc.) to account for with respect to later movements (such as head turns) that may impact directionality. In some embodiments, aspects of head position can be determined based on motion sensor data and/or components thereof such as a gyroscope. In some embodiments, aspects of head position can be determined and/or confirmed such as by using a camera associated with an accessory device. In some embodiments, an instruction can be issued by the system to the wearer to cause them to change their position (head, body, etc.) before the exercise or a particular exercise movement begins.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

It should be appreciated that the terms “vestibular retraining” may be used interchangeably with other similar terms such as “balance training”, “balance rehabilitation”, “vestibular rehabilitation”, “vestibular habituation”, and the like, without substantially deviating from the intended scope of the specification.

Vestibular retraining therapy (VRT) may be helpful to individuals with vestibular disorders or who often experience problems with vertigo, dizziness, visual disturbance, and/or imbalance/falls. VRT may be helpful when addressing problems secondary to vestibular disorders, such as nausea and/or vomiting, reduced ability to focus or concentrate, and fatigue. It will also be appreciated that balance problems may contribute to emotional problems such as anxiety and depression. Additionally, one of the consequences of having a vestibular disorder is that symptoms may cause the individual to adopt a sedentary lifestyle in order to avoid inducing, or worsening, dizziness and imbalance. As a result, secondary decreased muscle strength and flexibility, increased joint stiffness, and reduced stamina can be alleviated using vestibular rehabilitation exercises. Balance training may also be beneficial to individuals who may not experience appreciable balance deficits, such as athletes who wish to improve their balance abilities.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A vestibular rehabilitation management system comprising:
an ear-worn device comprising
a control circuit;
a microphone in communication with the control circuit; and
a motion sensor in communication with the control circuit;
wherein the vestibular rehabilitation management system is configured to
evaluate a wearer of the ear-worn device; and
select a vestibular training exercise based on the evaluation.

2. The vestibular rehabilitation management system of any of claims 1 and 3-50, wherein the vestibular training exercise selection includes selection of a dominant leg.

3. The vestibular rehabilitation management system of any of claims 1-2 and 4-50, wherein the vestibular training exercise selection includes selection of a vestibular training exercise difficulty.

4. The vestibular rehabilitation management system of any of claims 1-3 and 5-50, wherein the vestibular training exercise difficulty is selected with respect to a least one of a strength intensity index value, a vestibular function intensity index value, postural stability intensity index value, a reaction speed intensity index value, a stamina intensity index value, a speed intensity index value, a visual intensity index value, an environmental stimulation intensity index value, a proprioception intensity index value, and a cognitive load intensity index value.

5. The vestibular rehabilitation management system of any of claims 1-4 and 6-50, wherein the vestibular rehabilitation management system is configured to select a vestibular training exercise based on a mode setting stored or accessed by the vestibular rehabilitation management system.

6. The vestibular rehabilitation management system of any of claims 1-5 and 7-50, wherein the mode setting is selected from the group consisting of a training mode, a maintenance mode, and a daily life mode.

7. The vestibular rehabilitation management system of any of claims 1-6 and 8-50, wherein the vestibular rehabilitation management system is configured to evaluate the wearer of the ear-worn device by sensing their movement over a time period preceding selection of the vestibular training exercise.

8. The vestibular rehabilitation management system of any of claims 1-7 and 9-50, wherein movement is analyzed to detect at least one of swaying, stumbles, balance recovery, falls, and near-falls.

9. The vestibular rehabilitation management system of any of claims 1-8 and 10-50, wherein the time period preceding selection of the vestibular training exercise spans about 1 minute to 4 weeks.

10. The vestibular rehabilitation management system of any of claims 1-9 and 11-50, wherein the time period preceding selection of the vestibular training exercise includes a time period wherein the wearer of the ear-worn device is performing an exercise.

11. The vestibular rehabilitation management system of any of claims 1-10 and 12-50, wherein no vestibular training exercise is selected if detected movement crosses a threshold value.

12. The vestibular rehabilitation management system of any of claims 1-11 and 13-50, wherein the vestibular rehabilitation management system is further configured to present the selected vestibular training exercise to the wearer of the ear-worn device as a suggestion.

13. The vestibular rehabilitation management system of any of claims 1-12 and 14-50, wherein the vestibular rehabilitation management system selects a time to present the selected vestibular training exercise to the wearer of the ear-worn device as a suggestion.

14. The vestibular rehabilitation management system of any of claims 1-13 and 15-50, wherein the time is based on detected movement patterns of the wearer of the ear-worn device.

15. The vestibular rehabilitation management system of any of claims 1-14 and 16-50, wherein the vestibular rehabilitation management system is further configured to present the selected vestibular training exercise to a care provider for the wearer of the ear-worn device as a suggestion.

16. The vestibular rehabilitation management system of any of claims 1-15 and 17-50, wherein the vestibular rehabilitation management system is configured to evaluate the wearer of the ear-worn device by sensing their ability to complete a preceding exercise.

17. The vestibular rehabilitation management system of any of claims 1-16 and 18-50, wherein the vestibular rehabilitation management system is configured to evaluate the wearer of the ear-worn device by sensing an accuracy of completion of a preceding exercise.

18. The vestibular rehabilitation management system of any of claims 1-17 and 19-50, wherein the vestibular rehabilitation management system is configured to evaluate the wearer of the ear-worn device by determining the wearer's performance improvement over a series of preceding exercises.

19. The vestibular rehabilitation management system of any of claims 1-18 and 20-50, wherein the vestibular rehabilitation management system is configured to evaluate the wearer of the ear-worn device by evaluating a health record input.

20. The vestibular rehabilitation management system of any of claims 1-19 and 21-50, wherein the vestibular rehabilitation management system is configured to evaluate the wearer of the ear-worn device by evaluating consumption of a medication.

21. The vestibular rehabilitation management system of any of claims 1-20 and 22-50, wherein the vestibular rehabilitation management system is configured to evaluate the wearer of the ear-worn device by matching at least one of their age, gender, sex at birth, size or medical record with records stored in a database.

22. The vestibular rehabilitation management system of any of claims 1-21 and 23-50, wherein the vestibular rehabilitation management system is configured to evaluate the wearer of the ear-worn device by evaluating answers to one or more questions posed to the ear-worn device wearer.

23. The vestibular rehabilitation management system of any of claims 1-22 and 24-50, wherein the vestibular rehabilitation management system is configured to evaluate the wearer of the ear-worn device by evaluating measuring a degree of head or body sway with planar specificity.

24. The vestibular rehabilitation management system of any of claims 1-23 and 25-50, wherein the vestibular rehabilitation management system is configured to evaluate the wearer of the ear-worn device by evaluating maximum lean before imbalance occurs.

25. The vestibular rehabilitation management system of any of claims 1-24 and 26-50, wherein the vestibular rehabilitation management system is configured to evaluate the wearer of the ear-worn device by measuring a reaction speed of the wearer of the ear-worn device.

26. The vestibular rehabilitation management system of any of claims 1-25 and 27-50, wherein the vestibular rehabilitation management system is configured to evaluate the wearer of the ear-worn device by evaluating imbalance recovery.

27. The vestibular rehabilitation management system of any of claims 1-26 and 28-50, wherein the vestibular rehabilitation management system is configured to evaluate the wearer of the ear-worn device by evaluating imbalance recovery speed.

28. The vestibular rehabilitation management system of any of claims 1-27 and 29-50, wherein evaluating imbalance recovery includes measuring a degree of head or body sway after imbalance.

29. The vestibular rehabilitation management system of any of claims 1-28 and 30-50, wherein evaluating imbalance recovery includes measuring foot impact magnitude after imbalance.

30. The vestibular rehabilitation management system of any of claims 1-29 and 31-50, wherein evaluating imbalance recovery includes measuring foot speed after imbalance.

31. The vestibular rehabilitation management system of any of claims 1-30 and 32-50, wherein the vestibular rehabilitation management system is configured to evaluate the wearer of the ear-worn device by initiating imbalance and then evaluating imbalance recovery.

32. The vestibular rehabilitation management system of any of claims 1-31 and 33-50, wherein the vestibular rehabilitation management system is configured to evaluate the wearer of the ear-worn device by detecting imbalance and then evaluating imbalance recovery.

33. The vestibular rehabilitation management system of any of claims 1-32 and 34-50, further comprising an ear-worn device monitoring system in communication with the ear-worn device, the ear-worn device monitoring system comprising a control circuit.

34. The vestibular rehabilitation management system of any of claims 1-33 and 35-50, wherein the vestibular rehabilitation management system is configured to receive information from an accessory device.

35. The vestibular rehabilitation management system of any of claims 1-34 and 36-50, the information received from the accessory device comprising movement data regarding the wearer of the ear-worn device.

36. The vestibular rehabilitation management system of any of claims 1-35 and 37-50, wherein the movement data from the accessory device reflects movement of at least one of the wearer's arm, leg, or hip.

37. The vestibular rehabilitation management system of any of claims 1-36 and 38-50, wherein movement data below a threshold value is indicative of the accessory device not being held.

38. The vestibular rehabilitation management system of any of claims 1-37 and 39-50, wherein the vestibular rehabilitation management system is configured to record patterns of the accessory device being held and not being held.

39. The vestibular rehabilitation management system of any of claims 1-38 and 40-50, wherein the vestibular rehabilitation management system is configured to pace exercises based on patterns of the accessory device being held and not being held.

40. The vestibular rehabilitation management system of any of claims 1-39 and 41-50, the information received from the accessory device comprising data derived from a camera.

41. The vestibular rehabilitation management system of any of claims 1-40 and 42-50, the information received from the accessory device comprising data regarding whether the wearer of the ear-worn device is using their arm for support.

42. The vestibular rehabilitation management system of any of claims 1-41 and 43-50, the accessory device comprising a personal communications device.

43. The vestibular rehabilitation management system of any of claims 1-42 and 44-50, the accessory device comprising a smart phone.

44. The vestibular rehabilitation management system of any of claims 1-43 and 45-50, wherein the vestibular rehabilitation management system is configured to receive information regarding an environment of the wearer of the ear-worn device from an accessory device.

45. The vestibular rehabilitation management system of any of claims 1-44 and 46-50, the information regarding the environment of the wearer comprising information regarding obstacles in the environment of the wearer of the ear-worn device.

46. The vestibular rehabilitation management system of any of claims 1-45 and 47-50, the ear-worn device further comprising a blood pressure sensor.

47. The vestibular rehabilitation management system of any of claims 1-46 and 48-50, the ear-worn device further comprising a heart rate sensor.

48. The vestibular rehabilitation management system of any of claims 1-47 and 49-50, wherein the ear-worn device is further configured to transmit data based on motion sensor data to an accessory device.

49. The vestibular rehabilitation management system of any of claims 1-48 and 50, wherein the ear-worn device comprises a hearing aid.

50. The vestibular rehabilitation management system of any of claims 1-49, further comprising a second ear-worn device.

51. A vestibular rehabilitation management system comprising:
an ear-worn device comprising
a control circuit;
a microphone in communication with the control circuit; and
a motion sensor in communication with the control circuit;
wherein the ear-worn device is configured to
monitor signals from the motion sensor to detect system declared performance of an exercise;
monitor signals from the microphone to detect wearer declared performance of an exercise; and
compare system declared performance of the exercise with wearer declared performance of the exercise.

52. The vestibular rehabilitation management system of any of claims 51 and 53-54, the vestibular rehabilitation management system configured to initiate a report to a third party if the system declared performance of the exercise and the wearer declared performance of the exercise deviates by at least a threshold value.

53. The vestibular rehabilitation management system of any of claims 51-52 and 54, the vestibular rehabilitation management system configured to initiate a calibration process if the system declared performance of the exercise and the wearer declared performance of the exercise deviates by at least a threshold value.

54. The vestibular rehabilitation management system of any of claims 51-53, wherein the system declared performance of an exercise is based on motion sensor signals crossing a threshold value.

55. A vestibular rehabilitation management system comprising:
an ear-worn device comprising
a control circuit;
a microphone in communication with the control circuit; and
a motion sensor in communication with the control circuit;
wherein the vestibular rehabilitation management system is configured to
monitor signals from the microphone to detect a voice of a wearer of the ear-worn device.

56. The vestibular rehabilitation management system of any of claims 55 and 57-63, wherein the vestibular rehabilitation management system is further configured to evaluate the signals from the microphone to detect spoken commands.

57. The vestibular rehabilitation management system of any of claims 55-56 and 58-63, the spoken commands selected from the group consisting of start exercise, pause exercise, stop exercise, and proceed with next exercise.

58. The vestibular rehabilitation management system of any of claims 55-57 and 59-63, wherein the vestibular rehabilitation management system is further configured to evaluate the signals from the microphone to detect spoken commands and evaluate signals from the motion sensor to detect gesture commands.

59. The vestibular rehabilitation management system of any of claims 55-58 and 60-63, wherein the vestibular rehabilitation management system is further configured to evaluate the signals from the microphone to detect a condition of the wearer of the ear-worn device.

60. The vestibular rehabilitation management system of any of claims 55-59 and 61-63, wherein the vestibular rehabilitation management system is configured to select a vestibular training exercise based on the condition of the wearer of the ear-worn device.

61. The vestibular rehabilitation management system of any of claims 55-60 and 62-63, wherein the vestibular rehabilitation management system is further configured to evaluate the signals from the microphone to detect an exertion state of the wearer of the ear-worn device.

62. The vestibular rehabilitation management system of any of claims 55-61 and 63, wherein the vestibular rehabilitation management system is further configured to evaluate the signals from the microphone to detect a quality of speech of the wearer of the ear-worn device.

63. The vestibular rehabilitation management system of any of claims 55-62, wherein the vestibular rehabilitation management system is further configured to evaluate the signals from the microphone to detect a distress state of the wearer of the ear-worn device.

64. A vestibular rehabilitation management system comprising:
an ear-worn device comprising
a control circuit;
a microphone in communication with the control circuit; and
a sensor package in communication with the control circuit;
wherein the vestibular rehabilitation management system is configured to
initiate or detect performance of an exercise by a wearer of the ear-worn device;
monitor signals from the sensor package to detect a performance status of an exercise; and
send information regarding the exercise to a remote location.

65. The vestibular rehabilitation management system of any of claims 64 and 66-67, wherein the vestibular rehabilitation management system is further configured to initiate a real-time connection with a third party.

66. The vestibular rehabilitation management system of any of claims 64-65 and 67, wherein the vestibular rehabilitation management system is further configured to receive input from a third party regarding the exercise or a future exercise.

67. The vestibular rehabilitation management system of any of claims 64-66, wherein the vestibular rehabilitation management system sends information regarding the exercise to the remote location if the performance status represents a change over a previous performance status crossing a threshold value.

68. A vestibular rehabilitation management system comprising:
a first ear-worn device comprising
a first control circuit;
a first power control circuit in communication with the first control circuit, the first power control circuit comprising
a first battery;
a first microphone in communication with the first control circuit; and
a first motion sensor in communication with the first control circuit; and
a second ear-worn device comprising
a second control circuit;
a second power control circuit in communication with the second control circuit, the second power control circuit comprising
a second battery;
a second microphone in communication with the first control circuit; and
a second motion sensor in communication with the first control circuit; and
wherein the vestibular rehabilitation management system is configured to initiate or detect initiation of an exercise;
turn on the first motion sensor and monitor a signal from the first motion sensor;
turn off the first motion sensor, turn on the second motion sensor, and monitor a signal from the second motion sensor.

69. The vestibular rehabilitation management system of any of claims 68 and 70-71, wherein the vestibular rehabilitation management system is configured to turn off the first motion sensor, turn on the second motion sensor, and monitor a signal from the second motion sensor based on a time schedule.

70. The vestibular rehabilitation management system of any of claims 68-69 and 71, wherein the vestibular rehabilitation management system is configured to turn off the first motion sensor, turn on the second motion sensor, and monitor a signal from the second motion sensor based on a comparison of a remaining stored charge of the first battery versus a remaining stored charge of the second battery.

71. The vestibular rehabilitation management system of any of claims 68-70, the first motion sensor and the second motion sensor comprising a gyroscope.

72. A vestibular rehabilitation management system comprising:
a first ear-worn device comprising
a first control circuit;
a first power control circuit in communication with the first control circuit, the first power control circuit comprising
a first battery;
a first microphone in communication with the first control circuit; and
a first motion sensor in communication with the first control circuit; and
a second ear-worn device comprising
a second control circuit;
a second power control circuit in communication with the second control circuit, the second power control circuit comprising
a second battery;
a second microphone in communication with the first control circuit; and
a second motion sensor in communication with the first control circuit; and
wherein the vestibular rehabilitation management system is configured to initiate or detect initiation of an exercise;
select either the first ear-worn device or the second ear-worn device;
turn on the motion sensor of the selected device and/or execute wireless radio operations with the selected device;
turn off the motion sensor of the non-selected device and/or cease or reduce wireless radio operations of the non-selected device.
